(19) United States
(12) Patent Application Publication
(10) Pub. No.: US 2019/0284274 A1
(43) Pub. Date: Sep. 19, 2019

(54) ERFE SPECIFIC ANTIBODIES COMPOSITIONS AND METHODS OF USE

(71) Applicant: Silarus Therapeutics, Inc., La Jolla, CA (US)

(72) Inventors: Xin DU, La Jolla, CA (US); Justin CHAPMAN, San Diego, CA (US); Marc NASOFF, Rancho Santa Fe, CA (US); Tina Michele SACH, San Diego, CA (US); Vanessa CICCHINI, San Diego, CA (US); Hua WU, San Diego, CA (US)

(21) Appl. No.: 16/322,889

(22) PCT Filed: Aug. 4, 2017
(86) PCT No.: PCT/US2017/045606

§ 371 Date: Feb. 1, 2019

Related U.S. Application Data
(60) Provisional application No. 62/371,478, filed on Aug. 5, 2016.

(51) Int. Cl. C07K 16/26

(52) U.S. Cl. CPC C07K 16/26; C07K 2317/34; C07K 2317/21; A61K 2039/505; C07K 2317/76; C07K 2317/92; C07K 2317/33; C07K 2317/24

(57) ABSTRACT

Provided herein are ERFE specific antibodies, compositions and methods of use for detection of ERFE polypeptides.

### RELATED APPLICATIONS

This application is the U.S. National Phase entry of International Application No. PCT/US2017/045606, filed on Aug. 4, 2017, which claims the benefit of U.S. Provisional Application No. 62/371,478, filed on Aug. 5, 2016, the contents of each are incorporated herein by reference in their entireties.

### SEQUENCE LISTING

The present application is accompanied by a Sequence Listing submitted electronically in ASCII format and which is incorporated by reference in its entirety. Said ASCII copy, created on Jul. 19, 2017, is named 45543-702_601_SL.txt and is 8,263 bytes in size.

### SUMMARY OF INVENTION

Provided herein, in certain embodiments, are isolated and purified antibodies that specifically bind to an epitope of an erythroferrone (ERFE) polypeptide. In some embodiments, the epitope of the ERFE polypeptide is on the N-terminus of ERFE. In some embodiments, the epitope of the ERFE polypeptide is at least 3 amino acids in length. In some embodiments, the epitope of the ERFE polypeptide comprises all or part of the sequence DPRDAWMLFV (SEQ ID NO: 1). In some embodiments, the epitope of the ERFE polypeptide comprises 3 to 6 amino acids within the sequence DPRDAWMLFV (SEQ ID NO: 1). In some embodiments, the epitope of the ERFE polypeptide comprises 4 to 6 amino acids within the sequence DPRDAWMLFV (SEQ ID NO: 1). In some embodiments, the epitope of the ERFE polypeptide comprises 5 to 6 amino acids within the sequence DPRDAWMLFV (SEQ ID NO: 1).

Also provided herein, in certain embodiments, are isolated and purified antibodies that bind to at least one of the following residues of ERFE: D77, P78, R79, D80, A81, W82, M83, L84, F85, or V86 of SEQ ID NO: 2. In some embodiments, the antibody blocks suppression of hepcidin mRNA expression by ERFE. In some embodiments, the antibody binds to at least D77. In some embodiments, the antibody binds to at least P78. In some embodiments, the antibody binds to at least R79. In some embodiments, the antibody binds to at least D80. In some embodiments, the antibody binds to at least A81. In some embodiments, the antibody binds to at least W82. In some embodiments, the antibody binds to at least M83. In some embodiments, the antibody binds to at least L84. In some embodiments, the antibody binds to at least F85. In some embodiments, the antibody binds to at least V86. In some embodiments, the antibody binds to at least two of the following residues of ERFE: D77, P78, R79, D80, A81, W82, M83, L84, F85, or V86 of SEQ ID NO: 2. In some embodiments, the antibody binds to at least three of the following residues of ERFE: D77, P78, R79, D80, A81, W82, M83, L84, F85, or V86 of SEQ ID NO: 2. In some embodiments, the antibody binds to at least four of the following residues of ERFE: D77, P78, R79, D80, A81, W82, M83, L84, F85, or V86 of SEQ ID NO: 2.

Also provided herein, in certain embodiments, are isolated and purified antibodies binding ERFE. In some embodiments, the antibody comprises an IgG constant domain. In some embodiments, the antibody comprises an IgG1, IgG2, IgG3, or IgG4 constant domain, or a variant thereof. In some embodiments, the antibody is a monoclonal antibody. In some embodiments, the antibody is an antigen binding fragment. In some embodiments, the antibody is a Fab fragment, F(ab′)2 fragment, single chain Fv (scFv), diabody, triabody, or minibody. In some embodiments, the antibody is human. In some embodiments, the antibody is humanized. In some embodiments, the antibody is chimeric. In some embodiments, the antibody partially or completely inhibits erythroferrone activity. In some embodiments, the antibody partially or completely inhibits suppression of hepcidin mRNA expression by ERFE.

Additionally provided herein, in certain embodiments, are host cells that produce isolated and purified antibodies binding ERFE disclosed herein. In some embodiments, the host cell is a mammalian cell. In some embodiments, the host cell is selected from the group consisting of CHO cells, ExpiCHO-S cells, CHO DG44 cells, CHO-K1 cells, myeloma cells, hybridoma cells, NSO cells, GS-NSO cells, HEK293 cells, HEK293T cells, HEK293E cells, HEK293-6E cells, HEK293F cells, and per.C6 cells. In some embodiments, the host cell is a CHO cell. In some embodiments, the host cell is a myeloma cell. In some embodiments, the host cell is a hybridoma. In some embodiments, the host cell is selected from the group consisting of E. coli cells, P. mirabilis cells, P. putidas cells, B. brevis cells, B. megaterium cells, B. subtilis cells, L. paracasei cells, S. lividans cells, Y. lipolytica cells, K. lactis cells, P. pastoris cells, S. cerevisiae cells, A. niger var. awamori cells, A. oryzae cells, L. tarentolae cells, T. ni larvae cells, S. frugiperda cells, Drosophila S2 cells, S. frugiperda SF9 cells, T. ni cells, and SfSWT-1 mimic cells. In some embodiments, the isolated and purified antibodies specifically bind to an epitope of an erythroferrone (ERFE) polypeptide. In some embodiments, the epitope of the ERFE polypeptide is on the N-terminus of ERFE. In some embodiments, the epitope of the ERFE polypeptide is at least 3 amino acids in length. In some embodiments, the epitope of the ERFE polypeptide comprises all or part of the sequence DPRDAWMLFV (SEQ ID NO: 1). In some embodiments, the epitope of the ERFE polypeptide comprises 3 to 6 amino acids within the sequence DPRDAWMLFV (SEQ ID NO: 1). In some embodiments, the epitope of the ERFE polypeptide comprises 4 to 6 amino acids within the sequence DPRDAWMLFV (SEQ ID NO: 1). In some embodiments, the epitope of the ERFE polypeptide comprises 5 to 6 amino acids within the sequence DPRDAWMLFV (SEQ ID NO: 1). In some embodiments, the isolated and purified antibodies bind to at least one of the following residues of ERFE: D77, P78, R79, D80, A81, W82, M83, L84, F85, or V86 of SEQ ID NO: 2, and blocks suppression of hepcidin mRNA expression by ERFE. In some embodiments, the antibody binds to at least D77. In some embodiments, the antibody binds to at least P78. In some embodiments, the antibody binds to at least R79. In some embodiments, the antibody binds to at least D80. In some embodiments, the antibody binds to at least A81. In some embodiments, the antibody binds to at least W82. In some embodiments, the antibody binds to at least M83. In some embodiments, the antibody binds to at least L84. In some embodiments, the antibody binds to at least F85. In some embodiments, the antibody binds to at least V86. In some embodiments, the antibody binds to at least two of the following residues of ERFE: D77, P78, R79, D80, A81, W82, M83, L84, F85, or V86 of SEQ ID NO: 2. In some embodiments, the antibody binds to at least three of the following residues of ERFE: D77, P78, R79, D80, A81, W82, M83, L84, F85, or V86 of SEQ ID NO: 2. In some embodiments, the antibody binds to at least four of the following residues of ERFE: D77, P78, R79, D80, A81, W82, M83, L84, F85, or V86 of SEQ ID NO: 2. In some embodiments, the antibody comprises an IgG constant domain. In some embodiments, the antibody comprises an IgG1, IgG2, IgG3, or IgG4 constant domain, or a variant thereof. In some embodiments, the antibody is a monoclonal antibody. In some embodiments, the antibody is an antigen binding fragment. In some embodiments, the antibody is a Fab fragment, F(ab′)2 fragment, single chain Fv (scFv), diabody, triabody, or minibody. In some embodiments, the antibody is human. In some embodiments, the antibody is humanized. In some embodiments, the antibody is chimeric. In some embodiments, the antibody partially or completely inhibits erythroferrone activity. In some embodiments, the antibody partially or completely inhibits suppression of hepcidin mRNA expression by ERFE.

Also provided herein, in certain embodiments, are compositions comprising isolated and purified antibodies binding ERFE disclosed herein and an excipient. In some embodiments, the isolated and purified antibodies specifically bind to an epitope of an erythroferrone (ERFE) polypeptide. In some embodiments, the epitope of the ERFE polypeptide is on the N-terminus of ERFE. In some embodiments, the epitope of the ERFE polypeptide is at least 3 amino acids in length. In some embodiments, the epitope of the ERFE polypeptide comprises all or part of the sequence DPRDAWMLFV (SEQ ID NO: 1). In some embodiments, the epitope of the ERFE polypeptide comprises 3 to 6 amino acids within the sequence DPRDAWMLFV (SEQ ID NO: 1). In some embodiments, the epitope of the ERFE polypeptide comprises 4 to 6 amino acids within the sequence DPRDAWMLFV (SEQ ID NO: 1). In some embodiments, the epitope of the ERFE polypeptide comprises 5 to 6 amino acids within the sequence DPRDAWMLFV (SEQ ID NO: 1). In some embodiments, the isolated and purified antibodies bind to at least one of the following residues of ERFE: D77, P78, R79, D80, A81, W82, M83, L84, F85, or V86 of SEQ ID NO: 2, and blocks suppression of hepcidin mRNA expression by ERFE. In some embodiments, the antibody binds to at least D77. In some embodiments, the antibody binds to at least P78. In some embodiments, the antibody binds to at least R79. In some embodiments, the antibody binds to at least D80. In some embodiments, the antibody binds to at least A81. In some embodiments, the antibody binds to at least W82. In some embodiments, the antibody binds to at least M83. In some embodiments, the antibody binds to at least L84. In some embodiments, the antibody binds to at least F85. In some embodiments, the antibody binds to at least V86. In some embodiments, the antibody binds to at least two of the following residues of ERFE: D77, P78, R79, D80, A81, W82, M83, L84, F85, or V86 of SEQ ID NO: 2. In some embodiments, the antibody binds to at least three of the following residues of ERFE: D77, P78, R79, D80, A81, W82, M83, L84, F85, or V86 of SEQ ID NO: 2. In some embodiments, the antibody binds to at least four of the following residues of ERFE: D77, P78, R79, D80, A81, W82, M83, L84, F85, or V86 of SEQ ID NO: 2. In some embodiments, the antibody comprises an IgG constant domain. In some embodiments, the antibody comprises an IgG1, IgG2, IgG3, or IgG4 constant domain, or a variant thereof. In some embodiments, the antibody is a monoclonal antibody. In some embodiments, the antibody is an antigen binding fragment. In some embodiments, the antibody is a Fab fragment, F(ab′)2 fragment, single chain Fv (scFv), diabody, triabody, or minibody. In some embodiments, the antibody is human. In some embodiments, the antibody is humanized. In some embodiments, the antibody is chimeric. In some embodiments, the antibody partially or completely inhibits erythroferrone activity. In some embodiments, the antibody partially or completely inhibits suppression of hepcidin mRNA expression by ERFE. In some embodiments, the antibody is a neutralizing antibody.

Also provided herein, in certain embodiments, are methods of producing an antibody that specifically binds to an ERFE polypeptide, comprising isolating and purifying an antibody from a host cell disclosed herein. In some embodiments, the isolated and purified antibodies specifically bind to an epitope of an erythroferrone (ERFE) polypeptide. In some embodiments, the epitope of the ERFE polypeptide is on the N-terminus of ERFE. In some embodiments, the epitope of the ERFE polypeptide is at least 3 amino acids in length. In some embodiments, the epitope of the ERFE polypeptide comprises all or part of the sequence DPRDAWMLFV (SEQ ID NO: 1). In some embodiments, the epitope of the ERFE polypeptide comprises 3 to 6 amino acids within the sequence DPRDAWMLFV (SEQ ID NO: 1). In some embodiments, the epitope of the ERFE polypeptide comprises 4 to 6 amino acids within the sequence DPRDAWMLFV (SEQ ID NO: 1). In some embodiments, the epitope of the ERFE polypeptide comprises 5 to 6 amino acids within the sequence DPRDAWMLFV (SEQ ID NO: 1). In some embodiments, the isolated and purified antibodies bind to at least one of the following residues of ERFE: D77, P78, R79, D80, A81, W82, M83, L84, F85, or V86 of SEQ ID NO: 2, and blocks suppression of hepcidin mRNA expression by ERFE. In some embodiments, the antibody binds to at least D77. In some embodiments, the antibody binds to at least P78. In some embodiments, the antibody binds to at least R79. In some embodiments, the antibody binds to at least D80. In some embodiments, the antibody binds to at least A81. In some embodiments, the antibody binds to at least W82. In some embodiments, the antibody binds to at least M83. In some embodiments, the antibody binds to at least L84. In some embodiments, the antibody binds to at least F85. In some embodiments, the antibody binds to at least V86. In some embodiments, the antibody binds to at least two of the following residues of ERFE: D77, P78, R79, D80, A81, W82, M83, L84, F85, or V86 of SEQ ID NO: 2. In some embodiments, the antibody binds to at least three of the following residues of ERFE: D77, P78, R79, D80, A81, W82, M83, L84, F85, or V86 of SEQ ID NO: 2. In some embodiments, the antibody binds to at least four of the following residues of ERFE: D77, P78, R79, D80, A81, W82, M83, L84, F85, or V86 of SEQ ID NO: 2. In some embodiments, the antibody comprises an IgG constant domain. In some embodiments, the antibody comprises an IgG1, IgG2, IgG3, or IgG4 constant domain, or a variant thereof. In some embodiments, the antibody is a monoclonal antibody. In some embodiments, the antibody is an antigen binding fragment. In some embodiments, the antibody is a Fab fragment, F(ab′)2 fragment, single chain Fv (scFv), diabody, triabody, or minibody. In some embodiments, the antibody is human. In some embodiments, the antibody is humanized. In some embodiments, the antibody is chimeric. In some embodiments, the antibody partially or completely inhibits erythroferrone activity. In some embodiments, the antibody partially or completely inhibits suppression of hepcidin mRNA expression by ERFE. In some embodiments, the antibody is a neutralizing antibody.

Also provided herein, in certain embodiments, are methods of modulating an ERFE polypeptide activity comprising contacting the ERFE polypeptide with a sufficient amount of an isolated and purified antibody binding ERFE provided herein or a composition thereof. In some embodiments, the isolated and purified antibodies specifically bind to an epitope of an erythroferrone (ERFE) polypeptide. In some embodiments, the epitope of the ERFE polypeptide is on the N-terminus of ERFE. In some embodiments, the epitope of the ERFE polypeptide is at least 3 amino acids in length. In some embodiments, the epitope of the ERFE polypeptide comprises all or part of the sequence DPRDAWMLFV (SEQ ID NO: 1). In some embodiments, the epitope of the ERFE polypeptide comprises 3 to 6 amino acids within the sequence DPRDAWMLFV (SEQ ID NO: 1). In some embodiments, the epitope of the ERFE polypeptide comprises 4 to 6 amino acids within the sequence DPRDAWMLFV (SEQ ID NO: 1). In some embodiments, the epitope of the ERFE polypeptide comprises 5 to 6 amino acids within the sequence DPRDAWMLFV (SEQ ID NO: 1). In some embodiments, the isolated and purified antibodies bind to at least one of the following residues of ERFE: D77, P78, R79, D80, A81, W82, M83, L84, F85, or V86 of SEQ ID NO: 2, and blocks suppression of hepcidin mRNA expression by ERFE. In some embodiments, the antibody binds to at least D77. In some embodiments, the antibody binds to at least P78. In some embodiments, the antibody binds to at least R79. In some embodiments, the antibody binds to at least D80. In some embodiments, the antibody binds to at least A81. In some embodiments, the antibody binds to at least W82. In some embodiments, the antibody binds to at least M83. In some embodiments, the antibody binds to at least L84. In some embodiments, the antibody binds to at least F85. In some embodiments, the antibody binds to at least V86. In some embodiments, the antibody binds to at least two of the following residues of ERFE: D77, P78, R79, D80, A81, W82, M83, L84, F85, or V86 of SEQ ID NO: 2. In some embodiments, the antibody binds to at least three of the following residues of ERFE: D77, P78, R79, D80, A81, W82, M83, L84, F85, or V86 of SEQ ID NO: 2. In some embodiments, the antibody binds to at least four of the following residues of ERFE: D77, P78, R79, D80, A81, W82, M83, L84, F85, or V86 of SEQ ID NO: 2. In some embodiments, the antibody comprises an IgG constant domain. In some embodiments, the antibody comprises an IgG1, IgG2, IgG3, or IgG4 constant domain, or a variant thereof. In some embodiments, the antibody is a monoclonal antibody. In some embodiments, the antibody is an antigen binding fragment. In some embodiments, the antibody is a Fab fragment, F(ab′)2 fragment, single chain Fv (scFv), diabody, triabody, or minibody. In some embodiments, the antibody is human. In some embodiments, the antibody is humanized. In some embodiments, the antibody is chimeric. In some embodiments, the antibody partially or completely inhibits erythroferrone activity. In some embodiments, the antibody partially or completely inhibits suppression of hepcidin mRNA expression by ERFE. In some embodiments, the antibody is a neutralizing antibody.

Additionally provided herein, in certain embodiments, are methods of detecting ERFE polypeptide in a sample comprising contacting the sample with an isolated and purified antibody binding ERFE provided herein and a detectable label. In some embodiments, the method comprises a sandwich ELISA. In some embodiments, the sandwich ELISA comprises incubating a well with a capture antibody, incubating a sample comprising the ERFE polypeptide in the well with the capture antibody, incubating a labeled detection antibody in the well with the ERFE polypeptide and the capture antibody and then measuring the amount of detection antibody bound to the ERFE and capture antibody. In some embodiments, the labeled detection antibody is biotinylated, fluorescent, or enzyme conjugated. In some embodiments, the sandwich ELISA comprises incubating a well with a capture antibody, incubating a sample comprising the ERFE polypeptide in the well with the capture antibody, incubating a biotinylated detection antibody in the well with the ERFE polypeptide and the capture antibody, incubating a streptavidin-HRP conjugate in the well with the biotinylated detection antibody, the ERFE polypeptide and the capture antibody, adding a substrate and measuring an absorbance value. In some embodiments, the substrate is colormetric, luminescent, or fluorescent. In some embodiments, the capture antibody binds to at least a portion of the C-terminus of an ERFE polypeptide and the detection antibody binds to at least one amino acid of SEQ ID NO: 1, wherein the capture antibody and the detection antibody are different antibodies. In some embodiments, the sample comprises blood, serum, urine, saliva, bone marrow, liver, spleen, cerebral spinal fluid, skeletal muscle, smooth muscle, adipose tissue, cells, or culture media. In some embodiments, the isolated and purified antibodies specifically bind to an epitope of an erythroferrone (ERFE) polypeptide. In some embodiments, the epitope of the ERFE polypeptide is on the N-terminus of ERFE. In some embodiments, the epitope of the ERFE polypeptide is at least 3 amino acids in length. In some embodiments, the epitope of the ERFE polypeptide comprises all or part of the sequence DPRDAWMLFV (SEQ ID NO: 1). In some embodiments, the epitope of the ERFE polypeptide comprises 3 to 6 amino acids within the sequence DPRDAWMLFV (SEQ ID NO: 1). In some embodiments, the epitope of the ERFE polypeptide comprises 4 to 6 amino acids within the sequence DPRDAWMLFV (SEQ ID NO: 1). In some embodiments, the epitope of the ERFE polypeptide comprises 5 to 6 amino acids within the sequence DPRDAWMLFV (SEQ ID NO: 1). In some embodiments, the isolated and purified antibodies bind to at least one of the following residues of ERFE: D77, P78, R79, D80, A81, W82, M83, L84, F85, or V86 of SEQ ID NO: 2, and blocks suppression of hepcidin mRNA expression by ERFE. In some embodiments, the antibody binds to at least D77. In some embodiments, the antibody binds to at least P78. In some embodiments, the antibody binds to at least R79. In some embodiments, the antibody binds to at least D80. In some embodiments, the antibody binds to at least A81. In some embodiments, the antibody binds to at least W82. In some embodiments, the antibody binds to at least M83. In some embodiments, the antibody binds to at least L84. In some embodiments, the antibody binds to at least F85. In some embodiments, the antibody binds to at least V86. In some embodiments, the antibody binds to at least two of the following residues of ERFE: D77, P78, R79, D80, A81, W82, M83, L84, F85, or V86 of SEQ ID NO: 2. In some embodiments, the antibody binds to at least three of the following residues of ERFE: D77, P78, R79, D80, A81, W82, M83, L84, F85, or V86 of SEQ ID NO: 2. In some embodiments, the antibody binds to at least four of the following residues of ERFE: D77, P78, R79, D80, A81, W82, M83, L84, F85, or V86 of SEQ ID NO: 2. In some embodiments, the antibody comprises an IgG constant domain. In some embodiments, the antibody comprises an IgG1, IgG2, IgG3, or IgG4 constant domain, or a variant thereof. In some embodiments, the antibody is a monoclonal antibody. In some embodiments, the antibody is an antigen binding fragment. In some embodiments, the antibody is a Fab fragment, F(ab′)2 fragment, single chain Fv (scFv), diabody, triabody, or minibody. In some embodiments, the antibody is human. In some embodiments, the antibody is humanized. In some embodiments, the antibody is chimeric. In some embodiments, the antibody partially or completely inhibits erythroferrone activity. In some embodiments, the antibody partially or completely inhibits suppression of hepcidin mRNA expression by ERFE. In some embodiments, the antibody is a neutralizing antibody.

Also provided herein, in certain embodiments, are kits comprising isolated and purified antibodies binding ERFE disclosed herein or compositions thereof, and at least one buffer. In some embodiments, at least one antibody is biotinylated. In some embodiments, the kit comprises a substrate. In some embodiments, the substrate is colormetric, luminescent, or fluorescent. In some embodiments, the isolated and purified antibodies specifically bind to an epitope of an erythroferrone (ERFE) polypeptide. In some embodiments, the epitope of the ERFE polypeptide is on the N-terminus of ERFE. In some embodiments, the epitope of the ERFE polypeptide is at least 3 amino acids in length. In some embodiments, the epitope of the ERFE polypeptide comprises all or part of the sequence DPRDAWMLFV (SEQ ID NO: 1). In some embodiments, the epitope of the ERFE polypeptide comprises 3 to 6 amino acids within the sequence DPRDAWMLFV (SEQ ID NO: 1). In some embodiments, the epitope of the ERFE polypeptide comprises 4 to 6 amino acids within the sequence DPRDAWMLFV (SEQ ID NO: 1). In some embodiments, the epitope of the ERFE polypeptide comprises 5 to 6 amino acids within the sequence DPRDAWMLFV (SEQ ID NO: 1). In some embodiments, the isolated and purified antibodies bind to at least one of the following residues of ERFE: D77, P78, R79, D80, A81, W82, M83, L84, F85, or V86 of SEQ ID NO: 2, and blocks suppression of hepcidin mRNA expression by ERFE. In some embodiments, the antibody binds to at least D77. In some embodiments, the antibody binds to at least P78. In some embodiments, the antibody binds to at least R79. In some embodiments, the antibody binds to at least D80. In some embodiments, the antibody binds to at least A81. In some embodiments, the antibody binds to at least W82. In some embodiments, the antibody binds to at least M83. In some embodiments, the antibody binds to at least L84. In some embodiments, the antibody binds to at least F85. In some embodiments, the antibody binds to at least V86. In some embodiments, the antibody binds to at least two of the following residues of ERFE: D77, P78, R79, D80, A81, W82, M83, L84, F85, or V86 of SEQ ID NO: 2. In some embodiments, the antibody binds to at least three of the following residues of ERFE: D77, P78, R79, D80, A81, W82, M83, L84, F85, or V86 of SEQ ID NO: 2. In some embodiments, the antibody binds to at least four of the following residues of ERFE: D77, P78, R79, D80, A81, W82, M83, L84, F85, or V86 of SEQ ID NO: 2. In some embodiments, the antibody comprises an IgG constant domain. In some embodiments, the antibody comprises an IgG1, IgG2, IgG3, or IgG4 constant domain, or a variant thereof. In some embodiments, the antibody is a monoclonal antibody. In some embodiments, the antibody is an antigen binding fragment. In some embodiments, the antibody is a Fab fragment, F(ab′)2 fragment, single chain Fv (scFv), diabody, triabody, or minibody. In some embodiments, the antibody is human. In some embodiments, the antibody is humanized. In some embodiments, the antibody is chimeric. In some embodiments, the antibody partially or completely inhibits erythroferrone activity. In some embodiments, the antibody partially or completely inhibits suppression of hepcidin mRNA expression by ERFE. In some embodiments, the antibody is a neutralizing antibody.

### BRIEF DESCRIPTION OF THE DRAWINGS

The novel features are set forth with particularity in the appended claims. A better understanding of the features and advantages herein will be obtained by reference to the following detailed description that sets forth illustrative embodiments, in which the principles herein are utilized, and the accompanying drawings of which:

FIG. 1 shows the results of an erythroferrone (ERFE) sandwich ELISA standard curve using recombinant ERFE.

FIG. 2 shows serum ERFE level in a time course after phlebotomy.

FIG. 3 shows serum ERFE level with a mouse model of β-thalassemia, th3/+ compared to ERFE KO/th3, ERFE KO, and wild type (WT).

FIG. 4 shows epitope mapping of the α-ERFE-1 antibody.

FIG. 5 shows epitope mapping of the α-ERFE-2 antibody.

FIG. 6 shows epitope mapping of the α-ERFE-3 antibody.

FIG. 7 shows antagonist activity of α-ERFE-1 antibody against an Fc-mERFE, a FlagHis-mERFE, and a FlagHis-hERFE.

FIG. 8 shows antagonist activity of α-ERFE-2 antibody against an Fc-mERFE, a FlagHis-mERFE, and a FlagHis-hERFE.

FIG. 9 shows binding kinetics of the α-ERFE-1 antibody binding to a biotinylated ERFE peptide (SEQ ID NO: 13).

FIG. 10: shows binding kinetics of the α-ERFE-2 antibody binding to a biotinylated ERFE peptide (SEQ ID NO: 13).

FIG. 11 shows a summary of anti-ERFE antibody binding data for various isolated and purified anti-ERFE antibodies

FIG. 12 shows functional anti-ERFE antibody screening via inhibition assay for isolated and purified anti-ERFE antibodies.

FIG. 13A shows an in vitro functional dose response for α-ERFE-1 in inhibiting ERFE-mediated suppression of HAMP.

FIG. 13B shows an in vitro functional dose response for α-ERFE-2 in inhibiting ERFE-mediated suppression of HAMP.

FIG. 13C shows an in vitro functional dose response for α-ERFE-1 and α-ERFE-3 in inhibiting ERFE-mediated suppression of HAMP.

FIG. 14A shows kinetic measurements of α-ERFE-1 antibody binding to monovalent human ERFE.

FIG. 14B shows kinetic measurements of α-ERFE-2 antibody binding to monovalent human ERFE.

FIG. 14C shows kinetic measurements of α-ERFE-3 antibody binding to monovalent human ERFE.

FIG. 15A shows a comparison of human and cyno ERFE binding to α-ERFE-1 antibody.

FIG. 15B shows a comparison of human and cyno ERFE binding to α-ERFE-2 antibody.

FIG. 16A shows a western blot of α-ERFE-land α-ERFE-2 specifically binding to human ERFE.

FIG. 16B shows a western blot of α-ERFE-1 for ERFE binding to spiked-in ERFE in Hep3B cell lysates.

FIG. 16C shows an ELISA which illustrates the specific binding of α-ERFE-1 and α-ERFE-2 to human ERFE but not to other family member CTRP proteins.

FIG. 17A shows in vivo activity of α-ERFE-1 and α-ERFE-2 in inhibiting changes to HAMP mRNA levels following ERFE stimulation by EPO.

FIG. 17B shows in vivo activity of α-ERFE-1 and α-ERFE-2 in inhibiting changes to serum Hepcidin levels following ERFE stimulation by EPO.

FIG. 17C shows in vivo activity of α-ERFE-1 and α-ERFE-2 in inhibiting changes to serum iron levels following ERFE stimulation by EPO.

FIG. 18A shows in vivo activity of α-ERFE-3 in inhibiting changes to HAMP mRNA levels following ERFE stimulation by EPO.

FIG. 18B shows in vivo activity of α-ERFE-3 in inhibiting changes to serum Hepcidin levels following ERFE stimulation by EPO.

FIG. 18C shows in vivo activity of α-ERFE-3 in inhibiting changes to serum iron levels following ERFE stimulation by EPO.

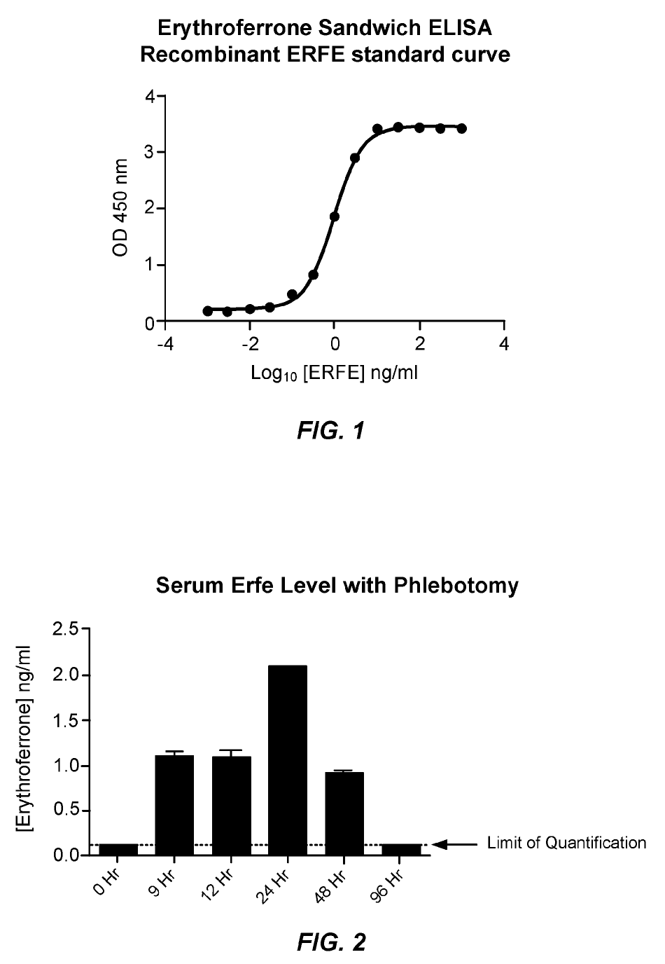

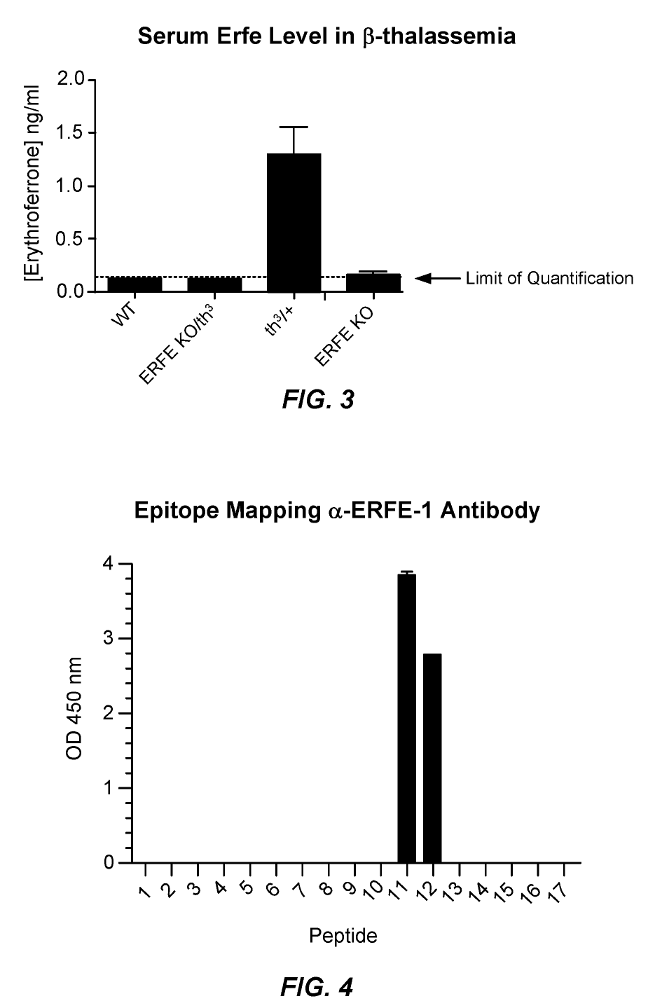

### DETAILED DESCRIPTION

Disclosed herein, in certain embodiments, are isolated and purified antibodies that specifically bind to an epitope of an erythroferrone (ERFE) polypeptide. Further disclosed herein, in certain embodiments, are isolated and purified antibodies that bind to at least one of the following residues of ERFE: D77, P78, R79, D80, A81, W82, M83, L84, F85, or V86 of SEQ ID NO: 2 and wherein the antibody blocks suppression of hepcidin mRNA expression by ERFE. Additionally disclosed herein, in certain embodiments, are host cells that produce an antibody that specifically binds to an epitope of an erythroferrone (ERFE) polypeptide sequence, for example an antibody that specifically binds to at least one of the following residues: D77, P78, R79, D80, A81, W82, M83, L84, F85, or V86 of SEQ ID NO: 2. Also disclosed herein, in certain embodiments, are compositions comprising an antibody that specifically binds to an epitope of an erythroferrone (ERFE) polypeptide sequence, for example an antibody that specifically binds to at least one of the following residues: D77, P78, R79, D80, A81, W82, M83, L84, F85, or V86 of SEQ ID NO: 2 and an excipient. Further disclosed herein, in certain embodiments, are methods of producing an antibody that specifically binds to an epitope of an erythroferrone (ERFE) polypeptide sequence, for example an antibody that specifically binds to at least one of the following residues: D77, P78, R79, D80, A81, W82, M83, L84, F85, or V86 of SEQ ID NO: 2, comprising isolating and purifying an antibody from the host cell. Additionally disclosed herein, in certain embodiments, are methods of modulating an ERFE polypeptide activity comprising contacting the ERFE polypeptide with a sufficient amount of an antibody that specifically binds to an epitope of an erythroferrone (ERFE) polypeptide sequence, for example an antibody that specifically binds to at least one of the following residues: D77, P78, R79, D80, A81, W82, M83, L84, F85, or V86 of SEQ ID NO: 2 or a composition thereof. Also disclosed herein, in certain embodiments, are methods of detecting ERFE polypeptide in a sample comprising contacting the sample with an antibody that specifically binds to an epitope of an erythroferrone (ERFE) polypeptide sequence, for example an antibody that specifically binds to at least one of the following residues: D77, P78, R79, D80, A81, W82, M83, L84, F85, or V86 of SEQ ID NO: 2 and a detectable label. Further disclosed herein, in certain embodiments, are kits comprising an antibody that specifically binds to an epitope of an erythroferrone (ERFE) polypeptide sequence, for example an antibody that specifically binds to at least one of the following residues: D77, P78, R79, D80, A81, W82, M83, L84, F85, or V86 of SEQ ID NO: 2 or a composition thereof, and at least one buffer.

### Definitions

As used herein, erythroferrone (including ERFE and Erfe) and its analogs and fragments are collectively referred to herein as “ERFE polypeptides”.

As used herein, “erythroferrone activity” refers to the ability of the substance to decrease hepatic hepcidin mRNA or serum hepcidin levels, or to increase serum iron levels, as compared to a control.

As used herein, the terms “protein”, “polypeptide” and “peptide” are used interchangeably to refer to two or more amino acids linked together.

As used herein, a “substantially purified” compound or a “isolated” compound, used interchangeably herein, refers to a compound that is removed from its natural environment and/or is at least about 60% free, about 75% free, about 90% free, or about 95-100% free from other macromolecular components or compounds with which the compound is associated with in nature or from its synthesis.

As used herein, “contacting,” when used in reference to a composition such as a protein (e.g., ERFE antibody), material, sample, or treatment, means a direct or indirect interaction between the composition (e.g., ERFE antibody) and the other referenced entity. A particular example of direct interaction is binding. A particular example of an indirect interaction is where the composition acts upon an intermediary molecule, which in turn acts upon the referenced entity. Thus, for example, contacting a cell (e.g., a hepatocyte) with an ERFE antibody includes allowing the antibody to bind to the cell (e.g., through binding to ERFE), or allowing the antibody to act upon an intermediary that in turn acts upon the cell.

The terms “assaying” and “measuring” and grammatical variations thereof are used interchangeably herein and refer to either qualitative or quantitative determinations, or both qualitative and quantitative determinations. When the terms are used in reference to binding, any means of assessing the relative amount, affinity, or specificity of binding is contemplated, including the various methods set forth herein and known in the art. For example, ERFE antibody binding to ERFE in some embodiments is assayed or measured by an ELISA assay.

As used herein, “neutralizing antibody” is an antibody that is capable of inhibiting a target protein. In some embodiments, an anti-ERFE neutralizing antibody reduces circulating ERFE levels. In some embodiments, an anti-ERFE neutralizing antibody reduces ERFE activity. In some embodiments, an anti-ERFE neutralizing antibody inhibits or prevents ERFE binding to a receptor.

As used herein, singular forms “a”, “and,” and “the” include plural referents unless the context clearly indicates otherwise. Thus, for example, reference to “an antibody” includes a plurality of antibodies and reference to “an antibody” in some embodiments includes multiple antibodies, and so forth.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

### Erythroferrone

Erythroferrone (ERFE) is a hormone that mediates between red blood cell production and the absorption and distribution of iron in individuals. ERFE is made in the marrow of an individual and its production is greatly increased when the production of red blood cells is stimulated, e.g., after bleeding or during recovery from anemia. ERFE regulates the supply of iron to meet the needs of red blood cell production in the marrow. Specifically, ERFE is found to act on the liver to suppress the production of the principal iron-regulatory protein, hepcidin. Thus, in certain instances, overproduction of ERFE causes iron overload in diseases such as β-thalassemia.

Hepcidin, a 25 amino acid peptide hormone synthesized by the liver, is the central regulator of iron homeostasis. Hepcidin acts by binding to the sole iron exporter ferroportin leading to its ubiquitination, internalization, and degradation in lysosomes. When ferroportin disappears from the cell membranes, dietary iron absorption is inhibited and recycled iron is sequestered in macrophages, decreasing iron availability for erythropoiesis. In contrast, low hepcidin allows ferroportin to remain active on cells that export iron to plasma, making more iron available for hemoglobin synthesis. Iron, inflammation, or ER stress stimulates hepcidin production, whereas hypoxia, iron deficiency, and increased erythropoietic activity repress it.

Hepcidin is suppressed after hemorrhage or erythropoietin (EPO) administration. Hepcidin is decreased in anemia caused by bleeding, hemolysis, or iron deficiency, or ineffective erythropoiesis. The suppressive effect of erythropoiesis on hepcidin is particularly prominent in diseases with ineffective erythropoiesis where erythrocyte precursors massively expand but mostly undergo apoptosis at the erythroblast stage rather than mature into erythrocytes.

ERFE is also referred to as Complement C1q tumor necrosis factor-related protein 15, Myonectin, FAM132B, C1QTNF15, and CTRP15. One non-limiting example of a full length human ERFE is a sequence set forth as:

(SEQ ID NO: 2)
MAPARRPAGARLLLVYAGLLAAAAAGLGSPEPGAPSRSRARREPPPGNEL
PRGPGESRAGPAARPPEPTAERAHSVDPRDAWMLFVRQSDKGVNGKKRSR
GKAKKLKFGLPGPPGPPGPQGPPGPIIPPEALLKEFQLLLKGAVRQRERA
EPEPCTCGPAGPVAASLAPVSATAGEDDDDVVGDVLALLAAPLAPGPRAP
RVEAAFLCRLRRDALVERRALHELGVYYLPDAEGAFRRGPGLNLTSGQYR
APVAGFYALAATLHVALGEPPRRGPPRPRDHLRLLICIQSRCQRNASLEA
IMGLESSSELFTISVNGVLYLQMGQWTSVFLDNASGCSLTVRSGSHFSAV
LLGV.

### ERFE Antibodies

Disclosed herein, in certain embodiments, are antibodies that specifically bind to an erythroferrone (ERFE) polypeptide. Further disclosed herein, in certain embodiments, are antibodies that bind to at least one of the following residues of ERFE: D77, P78, R79, D80, A81, W82, M83, L84, F85, or V86 of SEQ ID NO: 2. In some embodiments, the binding of the antibody to an ERFE polypeptide blocks suppression of hepcidin mRNA expression by ERFE. In some embodiments the antibody to an ERFE polypeptide is a neutralizing antibody.

In some embodiments, the antibodies disclosed herein that specifically bind to an ERFE polypeptide are isolated. In some embodiments, the antibodies that specifically bind to an ERFE polypeptide are substantially purified. In some embodiments, the antibodies that specifically bind to an ERFE polypeptide are isolated and substantially purified.

In some embodiments, the antibodies disclosed herein that specifically bind to an ERFE polypeptide are monoclonal antibodies. In some embodiments, the antibodies disclosed herein that specifically bind to an ERFE polypeptide are polyclonal antibodies. In some embodiments, the antibodies disclosed herein that specifically bind to an ERFE polypeptide are IgM antibodies, IgG antibodies, IgA antibodies, IgE antibodies, IgD antibodies, or any subclass thereof. In some embodiments, the antibodies disclosed herein that specifically bind to an erythroferrone (ERFE) polypeptide are IgM antibodies. In some embodiments, the antibodies that specifically bind to an erythroferrone (ERFE) polypeptide are IgG antibodies. In some embodiments, the antibodies that specifically bind to an erythroferrone (ERFE) polypeptide are IgA antibodies. In some embodiments, the antibodies that specifically bind to an erythroferrone (ERFE) polypeptide are IgE antibodies. In some embodiments, the antibodies that specifically bind to an erythroferrone (ERFE) polypeptide sequence are IgD antibodies.

In some embodiments, the antibodies that specifically bind to an erythroferrone (ERFE) polypeptide comprise an IgG constant domain, or variant thereof. In some embodiments, IgG constant domain variants herein comprise constant domains with reduced binding to complement proteins such as C1q. Some such variants with reduced binding to C1q include but are not limited to mutations at residues L235, G237, D270, N297, K322, P329, and P331 of IgG1; and N297, E318, K320, and K322 of IgG2. In some embodiments, IgG variants herein comprise constant domains having increased binding to FcRn. Some such variants with increased binding to FcRn include but are not limited to T250, M252, S254, T256, M428, H433, and N434. In additional embodiments, IgG variants herein include modifying an IgG2 Fc domain with amino acids from an IgG4 Fc domain to ablate effector function. In additional embodiments, IgG variants herein include modifying an IgG3 Fc domain with amino acids from an IgG1, IgG2, or IgG4 Fc domain. In some embodiments, the antibodies that specifically bind to an erythroferrone (ERFE) polypeptide comprise an IgG1, IgG2, IgG3, or IgG4 constant domain, or variant thereof. In some embodiments, the antibodies that specifically bind to an erythroferrone (ERFE) polypeptide are IgG1 antibodies. In some embodiments, the antibodies that specifically bind to an erythroferrone (ERFE) polypeptide are IgG2 antibodies. In some embodiments, the antibodies that specifically bind to an erythroferrone (ERFE) polypeptide are IgG3 antibodies. In some embodiments, the antibodies that specifically bind to an erythroferrone (ERFE) polypeptide are IgG4 antibodies.

In some embodiments, antibodies herein have kappa or lambda light chain sequences, either full length as in naturally occurring antibodies, mixtures thereof (i.e., fusions of kappa and lambda chain sequences), and subsequences/fragments thereof. Naturally occurring antibody molecules contain two kappa or two lambda light chains.

In some embodiments binding affinity is determined by association (Ka) and dissociation (Kd) rate. Equilibrium affinity constant, KD, is the ratio of Ka/Kd. In some embodiments, association (Ka) and dissociation (Kd) rates are measured using surface plasmon resonance (SPR). Instrumentation and methods for real time detection and monitoring of binding rates are known and are commercially available (BiaCore 2000, Biacore AB, ForteBio Octet). In some embodiments, KD values are defined as the ERFE antibody concentration required to saturate one half (50%) of the binding sites on ERFE.

In some embodiments, the antibodies that specifically bind to an erythroferrone (ERFE) polypeptide are antibody subsequences or antibody fragments. Antibody fragments include, but are not limited to, Fab, Fab′ and F(ab′)2, Fv, Fd, single-chain Fv (scFv), disulfide-linked Fvs (sdFv), Cov-X-Body, Diabody, Triabody, dsDb, DART, scDb, tandAbs, triple body, triple heads, Fab-scFv, Fab′)2-scFv2, dAb-CH1/CL, scFv4-Ig, IgG-scFv, scFv-IgG, DVD-Ig, IgG-sVD, sVD-IgG, 2 in 1-IgG, mAb2, Tandemab common LC, taFv-Fc, diabody, Di-diabody, scDbFc, scDb-CH3, scFv-Fc-scFV, HCAb-VHH, kih IgG, kih IgG common LC, scFv-kih-Fc, kih scFab-IgG, scFv-kih-CH3, CrossMab, mAb-Fv, kih-IgG-scFab, kih scFab-IgG-scFv, kih scFab-IgG-scFv,κ/λ-body common HC, SEED-body, CH3 charge pairs, hinge charge pairs, asymetric IgG, Duobody, nanobody, minibody, VL, and VH domain fragments.

In some embodiments, the antibody subsequences and antibody fragments have the binding affinity of a full length antibody, the binding specificity of a full length antibody, or one or more activities or functions of a full length antibody, e.g., a function or activity of ERFE antagonist or agonist antibody.

In some embodiments, the antibodies that specifically bind to an erythroferrone (ERFE) polypeptide are human. In some embodiments, the antibody is humanized. In some embodiments, the antibody is chimeric.

### ERFE Epitopes

In some embodiments, antibodies disclosed herein specifically binds to an epitope in an amino acid sequence of an ERFE polypeptide N-terminal sequence. In some embodiments, the epitope of the ERFE polypeptide is on the N-terminus of ERFE. In some embodiments, the epitope of the ERFE polypeptide is on the C-terminus of ERFE. In some embodiments, the epitope of the ERFE polypeptide is at least 3 amino acids in length.

In some embodiments, the epitope of the ERFE polypeptide to which an antibody disclosed herein binds comprises all or part of the sequence DPRDAWMLFV (SEQ ID NO: 1). In some embodiments, the epitope of the ERFE polypeptide comprises 3 to 6 amino acids within the sequence DPRDAWMLFV (SEQ ID NO: 1). In some embodiments, the epitope of the ERFE polypeptide comprises 4 to 6 amino acids within the sequence DPRDAWMLFV (SEQ ID NO: 1). In some embodiments, the epitope of the ERFE polypeptide comprises 5 to 6 amino acids within the sequence DPRDAWMLFV (SEQ ID NO: 1). In some embodiments, the antibody binds to at least D77 of SEQ ID NO: 2. In some embodiments, the antibody binds to at least P78 of SEQ ID NO: 2. In some embodiments, the antibody binds to at least R79 of SEQ ID NO: 2. In some embodiments, the antibody binds to at least D80 of SEQ ID NO: 2. In some embodiments, the antibody binds to at least A81 of SEQ ID NO: 2. In some embodiments, the antibody binds to at least W82 of SEQ ID NO: 2. In some embodiments, the antibody binds to at least M83 of SEQ ID NO: 2. In some embodiments, the antibody binds to at least L84 of SEQ ID NO: 2. In some embodiments, the antibody binds to at least F85 of SEQ ID NO: 2. In some embodiments, the antibody binds to at least V86 of SEQ ID NO: 2. In some embodiments, the antibody binds to at least two of the following residues of ERFE: D77, P78, R79, D80, A81, W82, M83, L84, F85, or V86 of SEQ ID NO: 2. In some embodiments, the antibody binds to at least three of the following residues of ERFE: D77, P78, R79, D80, A81, W82, M83, L84, F85, or V86 of SEQ ID NO: 2. In some embodiments, the antibody binds to at least four of the following residues of ERFE: D77, P78, R79, D80, A81, W82, M83, L84, F85, or V86 of SEQ ID NO: 2.

### Preparation of ERFE Antibodies

Further disclosed herein, in certain aspects, are methods of producing an antibody that specifically binds to an epitope of an erythroferrone (ERFE) polypeptide sequence, for example an antibody that specifically binds to at least one of the following residues: D77, P78, R79, D80, A81, W82, M83, L84, F85, or V86 of SEQ ID NO: 2. In some embodiments, the methods comprise isolating and purifying an antibody from the host cell.

In some embodiments, the antibodies disclosed herein comprise an antibody subsequence or fragment. In some embodiments, antibody subsequences and fragments are prepared by proteolytic hydrolysis of antibody, for example, by pepsin or papain digestion of whole antibodies. Antibody subsequences and fragments produced by enzymatic cleavage with pepsin provide a 5S fragment denoted F(ab′)2. In some embodiments, this fragment is further cleaved using a thiol reducing agent to produce 3.5S Fab′ monovalent fragments. Alternatively, an enzymatic cleavage using pepsin produces two monovalent Fab′ fragments and the Fe fragment directly. In some embodiments, other methods of cleaving antibodies, such as separation of heavy chains to form monovalent light-heavy chain fragments, further cleavage of fragments, or other enzymatic or chemical methods are also used.

In some embodiments, the antibodies disclosed herein comprise an antibody subsequence and fragment. For example, in some embodiments, VL or VH subsequences are joined by a linker sequence thereby forming a VL-VH chimera. In some embodiments, a combination of single-chain Fvs (scFv) subsequences is joined by a linker sequence thereby forming an scFv-scFv chimera. ERFE antibody subsequences and fragments include single-chain antibodies or variable region(s) alone or in combination with all or a portion of other ERFE antibody subsequences.

Antibodies, as well as subsequences and fragments thereof, are produced by genetic methodology. Techniques include expression of all or a part of the gene encoding the protein or antibody into a host cell such as Cos cells, CHO cells, ExpiCHO-S cells, CHO DG44 cells, CHO-K1 cells, myeloma cells, hybridoma cells, NSO cells, GS-NSO cells, HEK293 cells, HEK293T cells, HEK293E cells, HEK293-6E cells, HEK293F cells, per.C6 cells, myeloma cells, hybridoma cells, E. coli cells, P. mirabilis cells, P. putidas cells, B. brevis cells, B. megaterium cells, B. subtilis cells, L. paracasei cells, S. lividans cells, Y. lipolytica cells, K. lactis cells, P. pastoris cells, S. cerevisiae cells, A. niger var. awamori cells, A. oryzae cells, L. tarentolae cells, T. ni larvae cells, S. frugiperda cells, Drosophila S2 cells, S. frugiperda SF9 cells, T. ni cells, and SfSWT-1 mimic cells. In some embodiments, the host cell is a mammalian cell. In some embodiments, the host cell is a fungal cell. In some embodiments, the host cell is a bacterial cell. In some embodiments, the host cell is an insect cell. The recombinant host cells synthesize full length or a subsequence, for example, an scFv.

Modified forms of antibodies that specifically bind to an ERFE polypeptide include derivatized sequences, for example, amino acids in which free amino groups form amine hydrochlorides, p-toluene sulfonyl groups, carbobenzoxy groups; the free carboxy groups from salts, methyl and ethyl esters; free hydroxl groups that form O-acyl or O-alkyl derivatives, as well as naturally occurring amino acid derivatives, for example, 4-hydroxyproline, for proline, 5-hydroxylysine for lysine, homoserine for serine, ornithine for lysine, etc. In some embodiments, modifications are produced using methods known in the art.

Modified forms of antibodies that specifically bind to an ERFE polypeptide include additions and insertions. For example, in some embodiments, an addition is a covalent or non-covalent attachment of any type of molecule to the ERFE antibody. Typically, additions and insertions confer a distinct function or activity.

Additions and insertions include fusion (chimeric) antibodies, which have one or more molecules not normally present covalently attached to the antibody. A particular example is an amino acid sequence of another antibody to produce a multispecific antibody.

In some embodiments, antibodies disclosed herein are chimera or fusion antibodies with one or more additional domains covalently linked thereto to impart a distinct or complementary function or activity.

In some embodiments, antibodies disclosed herein comprise a heterologous domain. In some embodiments, heterologous domains are an amino acid addition or insertion, but are not restricted to amino acid residues. Thus, in some embodiments, a heterologous domain consists of any of a variety of different types of small or large functional moieties. Such moieties include nucleic acid, peptide, carbohydrate, lipid, or small organic compounds, such as a drug, metals (gold, silver), etc. Particular non-limiting examples of heterologous domains include, for example, tags, detectable labels and cytotoxic agents. Specific examples of tags and detectable labels include T7-, His-, myc-, HA- and FLAG-tags; enzymes (horseradish peroxidase, urease, catalase, alkaline phosphatase, beta-galactosidase, chloramphenicol transferase); enzyme substrates; ligands (e.g., biotin); receptors (avidin); radionuclides (e.g., C14, S35, P32, P33, H3, I125, and I131); electron-dense reagents; paramagnetic labels; fluorophores (fluorescein, rhodamine, phycoerthrin); chromophores; chemi-luminescent (imidazole, luciferase); and bio-luminescent agents. Specific examples of cytotoxic agents include diptheria, toxin, cholera toxin, and ricin.

In some embodiments, antibodies disclosed herein comprise a linker sequence that links to a first portion and a second portion of the antibody. In some embodiments, the linker enables the first portion of the antibody and the second portion of the antibody to maintain, at least in part, a distinct function or activity. In some embodiments, the linker sequence has a flexible structure, is cleavable (for example by a protease), is unable to form an ordered secondary structure, or has a hydrophobic or charged character. Amino acids typically found in flexible protein regions include glycine, asparagine and serine. Other near neutral amino acids, such as threonine and alanine, in some embodiments, are also used in the linker sequence. Linkers further include chemical cross-linking and conjugating agents, such as sulfo-succinimidyl derivatives (sulfo-SMCC, sulfo-SMPB), disuccinimidyl suberate (DSS), disuccinimidyl glutarate (DSG), and disuccinimidyl tartrate (DST).

In some embodiments, antibodies disclosed herein are glycosylated, acetylated, phosphorylated, amidated, formylated, ubiquitinatated, or derivatized by protecting or blocking groups and any of numerous chemical modifications. In some embodiments, antibodies disclosed herein comprise a lipid or a fatty acid. Other permutations and possibilities will be readily apparent to those of ordinary skill in the art, and are considered to be within the scope herein.

In some embodiments, antibodies disclosed herein are made using recombinant DNA technology via cell expression or in vitro translation. Antibodies disclosed herein are also produced by chemical synthesis using methods known in the art, for example, an automated peptide synthesis apparatus (see, e.g., Applied Biosystems, Foster City, Calif).

Any suitable method of producing polyclonal and monoclonal antibodies is contemplated for use herein. For example, ERFE or an immunogenic fragment thereof, optionally conjugated to a carrier such as keyhole limpet hemocyanin (KLH) or ovalbumin (e.g., BSA), or mixed with an adjuvant such as Freund's complete or incomplete adjuvant, and used to immunize an animal. In some embodiments, using hybridoma technology, splenocytes from immunized animals that respond to ERFE are isolated and fused with myeloma cells. Monoclonal antibodies produced by the hybridomas, in some embodiments, are screened for reactivity with ERFE or an immunogenic fragment thereof.

In some embodiments, animals that are immunized include primates, mice, rats, rabbits, goats, sheep, cattle, or guinea pigs. In some embodiments, initial and any optional subsequent immunization are through intravenous, intraperitoneal, intramuscular, or subcutaneous routes. Additionally, to increase the immune response, in some embodiments, antigen is coupled to another protein such as ovalbumin, keyhole limpet hemocyanin (KLH), thyroglobulin, or tetanus toxoid, or mixed with an adjuvant such as Freund's complete or incomplete adjuvant. In some embodiments, initial and any optional subsequent immunization is through intraperitoneal, intramuscular, intraocular, or subcutaneous routes. In some embodiments, subsequent immunizations are at the same or at different concentrations of ERFE preparation, and are at regular or irregular intervals.

Animals include those genetically modified to include human gene loci, which in some embodiments, are used to produce human antibodies. Using conventional hybridoma technology, in some embodiments, splenocytes from immunized mice that are high responders to the antigen are isolated and fused with myeloma cells. In some embodiments, a monoclonal antibody is obtained that binds to ERFE.

The term “human” when used in reference to an antibody, means that the amino acid sequence of the antibody is fully human, i.e., human heavy and human light chain variable and human constant regions. Thus, all of the amino acids are human or exist in a human antibody. An antibody that is non-human, in some embodiments, is made fully human by substituting the non-human amino acid residues with amino acid residues that exist in a human antibody. Amino acid residues present in human antibodies, CDR region maps and human antibody consensus residues are known in the art. Human antibodies therefore include antibodies in which one or more amino acid residues have been substituted with one or more amino acids present in any other human antibody.

ERFE antibodies include humanized antibodies, which in some embodiments, are produced using any suitable techniques including, for example, CDR-grafting; veneering or resurfacing; ; and chain shuffling. Human consensus sequences have previously used to produce humanized antibodies.

The term “humanized” when used in reference to an antibody, means that the amino acid sequence of the antibody has non-human amino acid residues (e.g., mouse, rat, goat, rabbit, etc.) of one or more complementarity determining regions (CDRs) that specifically bind to the desired antigen in an acceptor human immunoglobulin molecule, and one or more human amino acid residues in the Fv framework region (FR), which are amino acid residues that flank the CDRs. Antibodies referred to as “primatized” are within the meaning of “humanized” except that the acceptor human immunoglobulin molecule and framework region amino acid residues, in some embodiments, is any primate amino acid residue (e.g., ape, gibbon, gorilla, chimpanzees orangutan, macaque), in addition to any human residue. Human FR residues of the immunoglobulin, in some embodiments, are replaced with corresponding non-human residues. Residues in the CDR or human framework regions, in some embodiments, are therefore substituted with a corresponding residue from the non-human CDR or framework region donor antibody to alter antigen affinity or specificity, for example. A humanized antibody, in some embodiments, includes residues, which are found neither in the human antibody nor in the donor CDR or framework sequences. For example, in some embodiments, a FR substitution at a particular position that is not found in a human antibody or the donor non-human antibody is predicted to alter binding affinity or specificity of a human antibody at that position. Antibody framework and CDR substitutions based upon molecular modeling are well known in the art, e.g., by modeling of the interactions of the CDR and framework residues to identify framework residues important for antigen binding and sequence comparison to identify unusual framework residues at particular positions.

Antibodies that specifically bind to an ERFE polypeptide include chimeric antibodies. As used herein, the term “chimeric” and grammatical variations thereof, when used in reference to an antibody, means that the amino acid sequence of the antibody contains one or more portions that are derived from, obtained or isolated from, or based upon two or more different species. For example, in some embodiments, a portion of the antibody is human (e.g., a constant region) and another portion of the antibody is non-human (e.g., a murine heavy or murine light chain variable region). Thus, an example of a chimeric antibody is an antibody in which different portions of the antibody are of different species origins. Unlike a humanized or primatized antibody, a chimeric antibody, in some embodiments, has the different species sequences in any region of the antibody. Methods for producing chimeric antibodies are known in the art.

In some embodiments, antibodies that specifically bind to an ERFE polypeptide are also generated using hybridoma, recombinant, and phage display technologies, or a combination thereof.

In some embodiments, suitable techniques that additionally are employed in antibody methods include ERFE-based affinity purification, non-denaturing gel purification, HPLC or RP-HPLC, size exclusion, purification on protein A column, or any combination of these techniques. In some embodiments, ERFE antibody isotype is determined using an ELISA assay, for example in some embodiments, a human Ig is identified using mouse Ig-absorbed anti-human Ig.

Disclosed herein, in certain embodiments, are host cells that express an antibody, subsequence and fragment thereof that specifically binds to an ERFE polypeptide. In some embodiments, the host cell expresses an antibody that specifically binds an erythroferrone (ERFE) polypeptide. In some embodiments, a host cell disclosed herein expresses an antibody that binds to an epitope on the N-terminus of ERFE. In some embodiments, a host cell disclosed herein expresses an antibody that binds to all or part of the sequence DPRDAWMLFV (SEQ ID NO: 1). In some embodiments, a host cell disclosed herein expresses an antibody that binds to at least one of the following residues: D77, P78, R79, D80, A81, W82, M83, L84, F85, or V86 of SEQ ID NO: 2. In some embodiments, a host cell disclosed herein expresses an antibody that (a) binds to an epitope on the N-terminus of ERFE and (b) blocks suppression of hepcidin mRNA expression by ERFE. In some embodiments, a host cell disclosed herein expresses an antibody that (a) binds to all or part of the sequence DPRDAWMLFV (SEQ ID NO: 1) and (b) blocks suppression of hepcidin mRNA expression by ERFE. In some embodiments, a host cell disclosed herein expresses an antibody that (a) binds to at least one of the following residues: D77, P78, R79, D80, A81, W82, M83, L84, F85, or V86 of SEQ ID NO: 2 and (b) blocks suppression of hepcidin mRNA expression by ERFE. In some embodiments, the antibody is a neutralizing antibody.

In some embodiments, the host cell is a spleen cell, hybridoma cell, or CHO cell. The host cells, in some embodiments, are a plurality or population of cells from a primary cell isolate (e.g., splenocytes), a secondary or passaged cell isolate, or an established or immortalized cell culture (hybridoma or CHO cells). Host cells contemplated herein include but are not limited to Cos cells, CHO cells, ExpiCHO-S cells, CHO DG44 cells, CHO-K1 cells, myeloma cells, hybridoma cells, NSO cells, GS-NSO cells, HEK293 cells, HEK293T cells, HEK293E cells, HEK293-6E cells, HEK293F cells, per.C6 cells, myeloma cells, hybridoma cells, E. coli cells, P. mirabilis cells, P. putidas cells, B. brevis cells, B. megaterium cells, B. subtilis cells, L. paracasei cells, S. lividans cells, Y. lipolytica cells, K. lactis cells, P. pastoris cells, S. cerevisiae cells, A. niger var. awamori cells, A. oryzae cells, L. tarentolae cells, T. ni larvae cells, S. frugiperda cells, Drosophila S2 cells, S. frugiperda SF9 cells, T. ni cells, and SfSWT-1 mimic cells.

Further provided herein, in certain embodiments, are methods of producing antibodies that specifically bind to an ERFE polypeptide. In some embodiments, the method for producing an antibody that specifically binds to an erythroferrone (ERFE) polypeptide comprises administering a human ERFE, subsequence or fragment (e.g., an ERFE N-terminal sequence), optionally conjugated with human Fc recombinant protein, to an animal (e.g., a mouse), screening the animal for expression of an antibody binding to human ERFE, and selecting an animal that produces an antibody binding to human ERFE, isolating and culturing a population of cells expressing the antibody from the selected animal, and purifying the antibody from the cultured cells. In some embodiments, the method comprises determining whether the antibody has ERFE antagonist activity. In some embodiments, the method comprises determining whether the antibody is a neutralizing antibody. In some embodiments, the animal is a transgenic animal capable of producing human antibodies.

Additionally provided herein are methods of producing human antibodies that inhibit or prevent ERFE binding to a receptor. In some embodiments, the method for producing an antibody that binds to an erythroferrone (ERFE) polypeptide comprises administering a human ERFE, subsequence or fragment (e.g., an ERFE N-terminal sequence), optionally conjugated with human Fc recombinant protein, to an animal (e.g., a mouse), screening the animal for expression of an antibody binding to human ERFE, and selecting an animal that produces an antibody binding to human ERFE, isolating and culturing a population of cells expressing the antibody from the selected animal, and purifying the antibody from the cultured cells. In some embodiments, the method comprises determining whether the antibody inhibits or prevents ERFE binding to a receptor. In some embodiments, the method comprises determining whether the antibody is a neutralizing antibody.

Additionally provided herein are non-human transgenic animals that express an antibody having one or more of the following characteristics: a) is identical to an antibody produced by a hybridoma cell line; b) binds to an epitope in an amino acid sequence of ERFE N-terminal domain to which an antibody produced by a hybridoma cell line; c) has an ERFE binding affinity within about 1-5000 fold of an antibody produced by a hybridoma cell line; d) has an ERFE binding affinity within about KD 10⁻⁶ M to about KD 10⁻¹² M of an antibody produced by a hybridoma cell line; e) has the binding specificity of an antibody produced by a hybridoma cell line; or f) competes with an antibody produced by a hybridoma cell line for binding to ERFE.

Also provided herein, are methods of manufacturing an antibody that specifically binds to an ERFE polypeptide. In some embodiments, methods of manufacturing comprise culturing a host cell that expresses the antibody in a bioreactor or large culture vessel and purifying the antibody by methods known in the art. In some embodiments, the antibody is secreted into the culture media. In some embodiments, the antibody is not secreted into the culture media. In some embodiments, the antibody is purified by affinity chromatography. In some embodiments, the antibody is purified by anion exchange chromatography. In some embodiments, the antibody is purified by cation exchange chromatography. In some embodiments, the antibody is purified by size exclusion chromatography.

### ERFE Antibody Methods and Uses

Additionally disclosed herein, in certain embodiments, are methods of modulating an activity of an ERFE polypeptide comprising contacting the ERFE polypeptide with a sufficient amount of an antibody that specifically binds to an erythroferrone (ERFE) polypeptide, for example an antibody that specifically binds to at least one of the following residues: D77, P78, R79, D80, A81, W82, M83, L84, F85, or V86 of SEQ ID NO: 2 of an ERFE polypeptide, or a composition thereof. In some embodiments, the antibody is a neutralizing antibody.

Antibodies include antibodies that specifically bind to an ERFE polypeptide and modulate an ERFE function or activity in vivo or in vitro (e.g., in an individual). As used herein, the term “modulate” and grammatical variations thereof, when used in reference to an ERFE activity or function, means that the ERFE activity or function is detectably affected, altered or changed. Thus, an ERFE antibody that modulates an ERFE activity or function is an antibody that detectably affects, alters or changes one or more ERFE activities or functions, which, in some embodiments, includes, for example, binding of ERFE to an ERFE receptor, ERFE mediated signaling or an ERFE-mediated or ERFE-modulatable cell response, or another ERFE activity or function as set forth herein or otherwise known or knowable. In some embodiments, the ERFE antibody neutralizes ERFE activity. In some embodiments, the ERFE antibody is a neutralizing antibody. Detection of affected, altered, or changed ERFE activity is accomplished using in vitro, cell based, or in vivo ERFE assays. Such assays include but are not limited to hepcidin cellular expression assays, hepcidin in vivo assays, and blood iron level in vivo assays.

Antibodies binding to an ERFE polypeptide epitope comprising one or more amino acids of SEQ ID NO: 1, in some embodiments, have desirable properties, for example in inhibiting ERFE function or activity. In some embodiments, antibodies binding to at least one amino acid of SEQ ID NO: 1 inhibit ERFE function or activity. In some embodiments, antibodies not binding to at least one amino acid of SEQ ID NO: 1 do not inhibit ERFE function or activity.

In some embodiments, the antibody blocks partially or completely inhibits suppression of hepcidin mRNA expression by ERFE.

### ERFE Compositions

Also disclosed herein, in certain embodiments, are compositions comprising an antibody that specifically binds to an erythroferrone (ERFE) polypeptide sequence, for example an antibody that specifically binds to at least one of the following residues: D77, P78, R79, D80, A81, W82, M83, L84, F85, or V86 of SEQ ID NO: 2 of an ERFE polypeptide, and an excipient.

Disclosed herein, in certain embodiments, are compositions comprising (a) an antibody that specifically binds to an erythroferrone (ERFE) polypeptide sequence, and (b) a buffer or an excipient. Further disclosed herein, in some embodiments, are compositions comprising (a) an antibody that binds to at least one of the following residues: D77, P78, R79, D80, A81, W82, M83, L84, F85, or V86 of SEQ ID NO: 2, and (b) an excipient. In some embodiments, the antibody blocks suppression of hepcidin mRNA expression by ERFE. In some embodiments, the antibody is a neutralizing antibody.

In some embodiments, the composition is a solution, emulsion, dispersion media, coatings, and/or isotonic. In some embodiments, such formulations are contained in a liquid, emulsion, suspension, syrup, or elixir, or solid form, powder, granule, crystal, or microbead. In some embodiments, supplementary compounds (e.g., preservatives, antibacterial, antiviral and antifungal agents) are also incorporated into the composition.

In some embodiments, biodegradable, biocompatible polymers are used, such as ethylene vinyl acetate, polyanhydrides, polyglycolic acid, collagen, polyorthoesters, and polylactic acid. Methods for preparation of such formulations are known to those skilled in the art. In some embodiments, liposomal suspensions (including liposomes targeted to cells or tissues using antibodies or viral coat proteins) are also used as carriers.

### ERFE Assays

Also disclosed herein, in certain aspects, are methods of detecting an ERFE polypeptide in a sample comprising contacting the sample with an antibody that specifically binds to an erythroferrone (ERFE) polypeptide, for example an antibody that specifically binds to at least one of the following residues: D77, P78, R79, D80, A81, W82, M83, L84, F85, or V86 of SEQ ID NO: 2, and a detectable label.

Further provided herein, in certain aspects, are cell-free (e.g., in solution, in solid phase) and cell-based (e.g., in vitro or in vivo) methods of screening, detecting, and identifying ERFE. In some embodiments, the methods are performed in solution, in vitro using a biological material or sample, and in vivo, for example, a sample of cells or serum from an animal. In some embodiments, a method comprises contacting a biological material or sample with an antibody that binds to ERFE under conditions allowing binding of the antibody to ERFE; and assaying for binding of the antibody to ERFE. The binding of the antibody to ERFE detects the presence of ERFE. In some embodiments, ERFE is present on a cell or tissue. In some embodiments, ERFE is present in a sample of serum from an individual. In some embodiments, the biological material or sample is obtained from a mammalian individual.

Also provided herein, in some embodiments, are methods for detecting ERFE polypeptide comprising contacting a sample with an antibody described herein and a detectable label. In some embodiments, the method comprises a sandwich ELISA. In some embodiments, the sandwich ELISA comprises incubating a well with a capture antibody, incubating a sample comprising the ERFE polypeptide in the well with the capture antibody, incubating a labeled detection antibody in the well with the ERFE polypeptide and the capture antibody and then measuring the amount of detection antibody bound to the ERFE and capture antibody. In some embodiments, the sandwich ELISA comprises incubating a well with a capture antibody, incubating a sample comprising the ERFE polypeptide in the well with the capture antibody, incubating a biotinylated detection antibody in the well with the ERFE polypeptide and the capture antibody, incubating a streptavidin-HRP conjugate in the well with the biotinylated detection antibody, the ERFE polypeptide and the capture antibody, adding a substrate and measuring an absorbance value.

In some embodiments of the methods described herein the methods include samples comprising one or more of blood, serum, urine, saliva, bone marrow, liver, spleen, cerebral spinal fluid, skeletal muscle, smooth muscle, adipose tissue, cells, or culture media. In some embodiments, the method is fluorescence activated cell sorting (FACS), enzyme-linked immunosorbent assay (ELISA), Enzyme-Linked ImmunoSpot (ELISPOT), immunoprecipitation, Western blot, microscopy, competition assay, surface plasmon resonance (SPR), or radioimmunoassay (RIA). In some embodiments, the detectable label comprises an enzymatic label such as horseradish peroxidase (HRP), alkaline phosphatase (AP) or glucose oxidase; a fluorescent label such as Alexa Fluor® 350, Alexa Fluor® 405, Alexa Fluor® 488, Alexa Fluor® 532, Alexa Fluor® 546, Alexa Fluor® 555, Alexa Fluor® 568, Alexa Fluor® 594, Alexa Fluor® 647, Alexa Fluor® 680, Alexa Fluor® 750, BODIPY® FL, Coumarin, Cy®3, Cy®5, Fluorescein (FITC), Oregon Green®, Pacific Blue™, Pacific Green™, Pacific Orange™, Tetramethylrhodamine (TRITC), Texas Red®, or other fluorescent label; or a radioactive isotope such as 32P, 33P, 3H, 14C, 125I, or other radioactive isotope.

In additional embodiments disclosed herein, there are provided ELISA assays for detecting ERFE in a sample. In some embodiments, the ELISA assay comprises a sandwich ELISA assay. In particular aspects, the sandwich ELISA assay comprises a capture antibody binding to at least a portion of the C-terminus of an ERFE polypeptide and a detection antibody binding to at least one amino acid of SEQ ID NO: 1. In another aspect, the sandwich ELISA assay comprises a capture antibody binding to at least a portion of the N-terminus of an ERFE polypeptide and a detection antibody binding to at least one amino acid of SEQ ID NO: 1. In some embodiments, the capture antibody and the detection antibody are not the same antibody. In some embodiments, the labeled detection antibody is biotinylated, fluorescent, or enzyme conjugated. In some embodiments, the detection antibody is labeled with a label selected from the group consisting of horseradish peroxidase (HRP), alkaline phosphatase (AP) or glucose oxidase, Alexa Fluor® 350, Alexa Fluor® 405, Alexa Fluor® 488, Alexa Fluor® 532, Alexa Fluor® 546, Alexa Fluor® 555, Alexa Fluor® 568, Alexa Fluor® 594, Alexa Fluor® 647, Alexa Fluor® 680, Alexa Fluor® 750, BODIPY® FL, Coumarin, Cy®3, Cy®5, Fluorescein (FITC), Oregon Green®, Pacific Blue™, Pacific Green™, Pacific Orange™, Tetramethylrhodamine (TRITC), Texas Red®, 32P, 33P, 3H, 14C, and 125I. In some embodiments, the sandwich ELISA comprises an enzymatic substrate. In some embodiments, the enzymatic substrate comprises one or more of PNPP, ABTS, OPD, TMB, ONPG, CDP-Star, CSPD, DynaLight, SuperSignal ELISA Pico, SuperSignal ELISA Femto, QuantaBlu, Quanta Red, Amplex Red, or Amplex UltraRed. In some embodiments, the substrate is colormetric, luminescent, radioactive, or fluorescent. In some embodiments, the signal is detected by absorbance, luminescence, fluorescence, radiography, or scintillation counting.

### ERFE Antibody Kits

Further disclosed herein, in certain aspects, are kits comprising an antibody that specifically binds to an erythroferrone (ERFE) polypeptide, for example an antibody that specifically binds to at least one of the following residues: D77, P78, R79, D80, A81, W82, M83, L84, F85, or V86 of SEQ ID NO: 2 or a composition thereof, and at least one buffer. In some embodiments, the kit comprises at least one biotinylated antibody. In some embodiments, the kit comprises a substrate. In some embodiments, the substrate is colormetric, luminescent, or fluorescent.

Disclosed herein, in certain embodiments, are kits, comprising an isolated and purified antibody disclosed herein and at least one buffer, optionally in combination with instructions for using the kit components, e.g., instructions for performing a method herein. In some embodiments, the kit comprises an ERFE antibody, subsequence or fragment and instructions for detecting ERFE. For example, in some embodiments, the kits include reagents used for conducting assays, devices for obtaining samples to be assayed, devices for mixing reagents and conducting assays, and the like.

In some embodiments, the term “packaging material” refers to a physical structure housing the components of the kit. In some embodiments, the packaging material maintains the components sterilely, and is made of material commonly used for such purposes (e.g., paper, corrugated fiber, glass, plastic, foil, ampules, etc.). In some embodiments, the label or packaging insert includes appropriate written instructions. Instructions in some embodiments therefore include instructions for practicing any of the methods herein. Thus, in various embodiments, a kit includes a label or packaging insert including instructions for practicing a method herein in solution, in vitro, in vivo, or ex vivo.

In some embodiments, the instructions are on “printed matter,” e.g., on paper or cardboard within the kit, on a label affixed to the kit or packaging material, or attached to a vial or tube containing a component of the kit. In some embodiments instructions comprise audio or video medium and additionally are included on a computer readable medium, such as a disk (floppy diskette or hard disk), flash drive, optical CD such as CD- or DVD-ROM/RAM, magnetic tape, electrical storage media such as RAM and ROM and hybrids of these such as magnetic/optical storage media.

In some embodiments, kits herein additionally include a buffering agent, a preservative, or a stabilizing agent. In some embodiments, the kit includes control components for assaying for activity, e.g., a control sample or a standard. In some embodiments, each component of the kit is enclosed within an individual container or in a mixture and all of the various containers in certain embodiments are within single or multiple packages.

A number of embodiments have been described herein. Nevertheless, it will be understood that in some embodiments, various modifications are made without departing from the spirit and scope herein. Accordingly, the following examples are intended to illustrate but not limit the scope described in the claims.

### EXAMPLES

### Example 1

### ERFE Sandwich ELISA Protocol

96-well high binding plates (Costar #9018) were coated overnight at 4° C. with 100 ng/well anti-ERFE Capture Antibody in sodium carbonate buffer (50 mM pH 9.6) in 100 μl volume. The next morning, wells were washed three times with TBS-T, 350 μl per well. The plate was then blocked for 30 minutes at RT with SuperBlock T20 (Pierce #37536), 200 μl per well and washed one time with TBS-T, 350 μl per well. Next, samples or recombinant ERFE standard were diluted in Superblock T20 and incubated for 2 hours at RT, 100 μl per well. After 2 hours incubation, the plate was washed three times with TBS-T, 350 μl per well. Next, the plate was incubated for 1 hour with Biotinylated aERFE-1Detection Antibody diluted to 1 μg/ml in SuperBlock T20, 100 μl per well. After 1 hour incubation, the plate was washed three times with TBS-T 350 μl per well. Next the plate was incubated for 45 minutes with Streptavidin-HRP conjugate (Invitrogen #SNN2004) 1/5000 dilution in SuperBlock T20, 100 μl per well. After 45 minutes incubation, the plate was washed three times with TBS-T 350 μl per well. Next, Supersensitive Liquid Substrate TMB for ELISA (Sigma T4444), 100 μl per well, was added and color was allowed to develop. The reaction was stopped by adding 100 μl per well 1N H₂SO₄. Output was measured by O.D. determined at 450 nm.

### Example 2

### ERFE Antibody Epitope Mapping

Epitopes for ERFE antibodies were determined by ELISA assay. Briefly, polypeptides of 13 amino acids in length and 4 amino acids overlap spanning the N-terminal ERFE sequence were synthesized using standard solid phase chemistry and biotinylated. These peptides were purified to >75% purity and solubilized in double-distilled water or dimethyl sulfoxide (DMSO).

ELISA assay was then utilized to identify the corresponding epitope(s) for each antibody. 5 μg/mL of NeutrAvidin (Pierce, USA) in 50 mM pH 9.6 sodium carbonate/bicarbonate buffer was used to coat 96-well Corning high-binding EIA/RIA plates. The plates were washed three times with TBST buffer and blocked with Superblock T20 (Pierce, USA). One hundred microliters of the biotinylated peptides were diluted with PBS to 2 μg/mL and added to each well and incubated at room temperature for one hour. The plates were then washed again with TBST and purified antibody (2 μg/mL) diluted in Superblock T20 was added to the plates. After one hour room temperature incubation and washing steps, goat-anti-mouse IgG HRP-conjugated secondary antibody diluted in Superblock T20 was applied for one hour at room temperature. TMB was used as the HRP substrate for the detection.

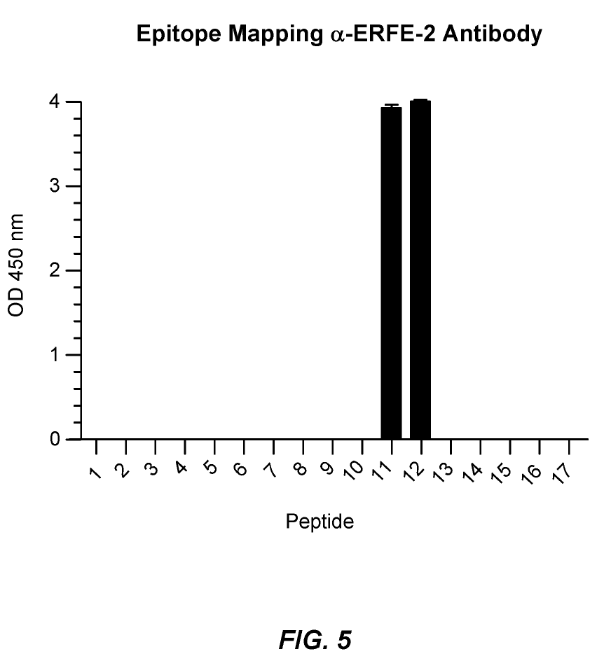

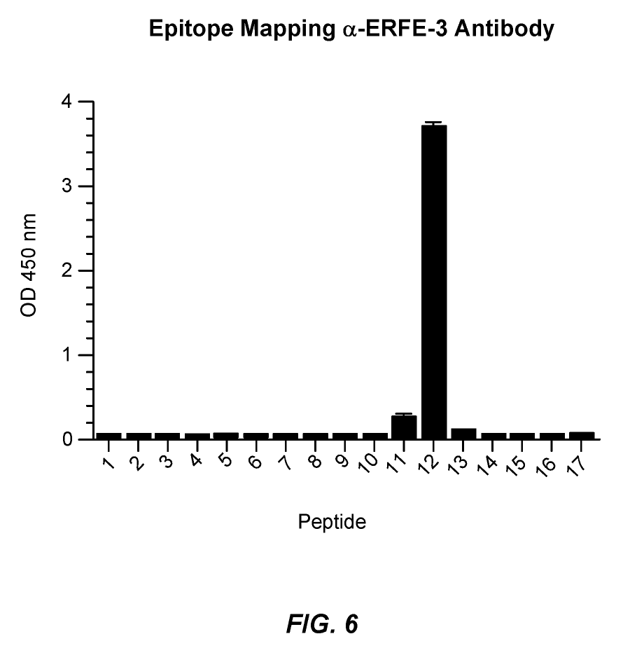

FIG. 4, FIG. 5, and FIG. 6 show that peptides 11 and 12 (SEQ ID NO: 13 and 14) were recognized by all three antibodies. The epitope, contained within the peptide DPRDAWMLFV (SEQ ID NO: 1) has 100% homology between mouse and human.

TABLE 1
ERFE peptides

| Peptide # | Sequence | SEQ ID NO: |
|---|---|---|
| 1 | GLGVPESAEPVGT | 3 |
| 2 | PESAEPVGTHARP | 4 |
| 3 | EPVGTHARPQPPG | 5 |
| 4 | THARPQPPGAELP | 6 |
| 5 | PQPPGAELPAPPA | 7 |
| 6 | GAELPAPPANSPP | 8 |
| 7 | PAPPANSPPEPTI | 9 |
| 8 | ANSPPEPTIAHAH | 10 |
| 9 | PEPTIAHAHSVDP | 11 |
| 10 | IAHAHSVDPRDAW | 12 |
| 11 | HSVDPRDAWMLFV | 13 |
| 12 | PRDAWMLFVKQSD | 14 |
| 13 | WMLFVKQSDKGIN | 15 |
| 14 | VKQSDKGINSKRR | 16 |
| 15 | DKGINSKRRSKAR | 17 |
| 16 | NSKRRSKARRLKL | 18 |
| 17 | RSKARRLKLGLPG | 19 |

### Example 3

### ERFE Antibodies Functional Activity

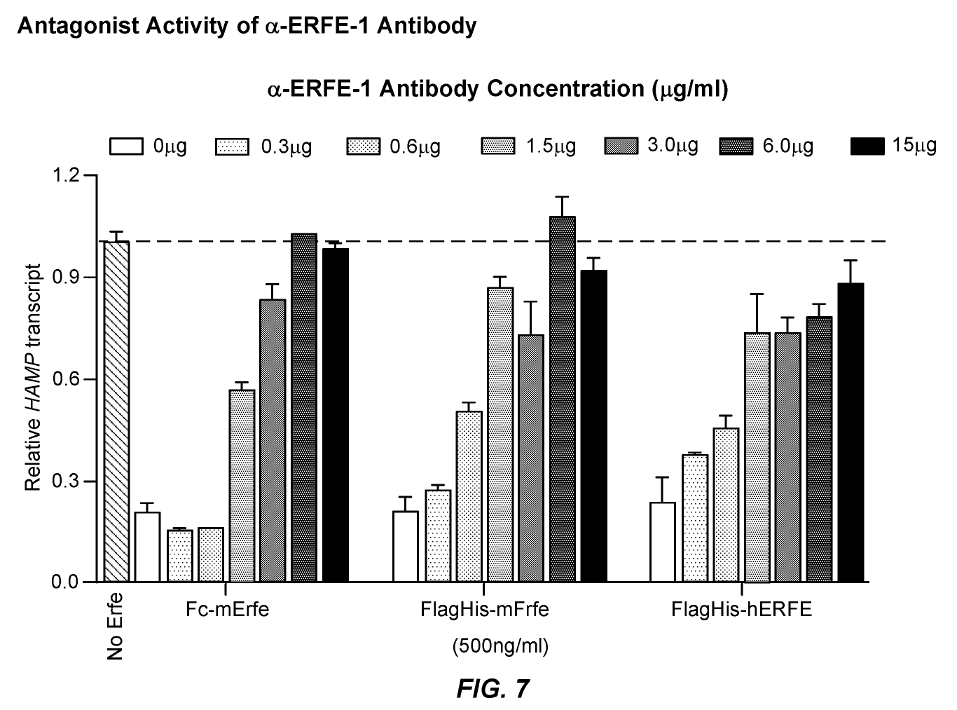

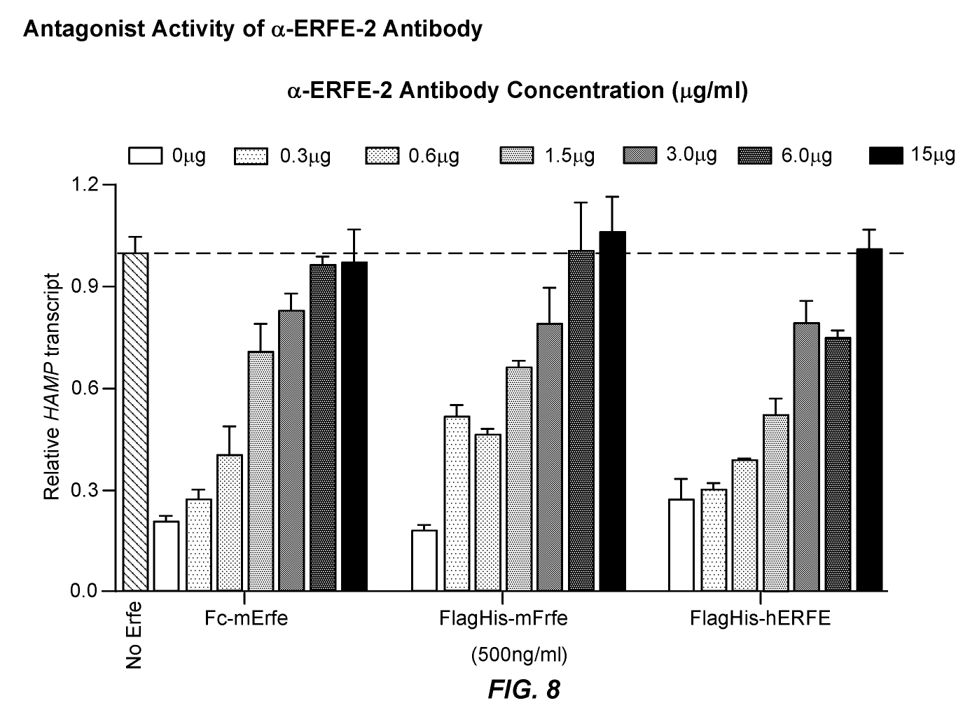

Functional activity was shown for ERFE antibodies in vitro using Hep3B cellular assays to characterize the neutralization of hERFE and mERFE driven suppression of hepcidin. For these assays ERFE or ERFE plus an antibody were pre-incubated for one hour at room temperature then added to a 24-well plate where 8×10⁴ Hep3B cells were seeded at approximately 70% confluency. The cells were incubated for six hours then lysed for total RNA extraction. One-step qRT-PCR was used to measure HAMP transcript levels relative to the HPRT1 gene. Results for these assays testing α-ERFE-1 and α-ERFE-2 antibodies are shown in FIG. 7 and FIG. 8, respectively.

### Example 4

### ERFE Antibodies Binding Kinetics Measurements

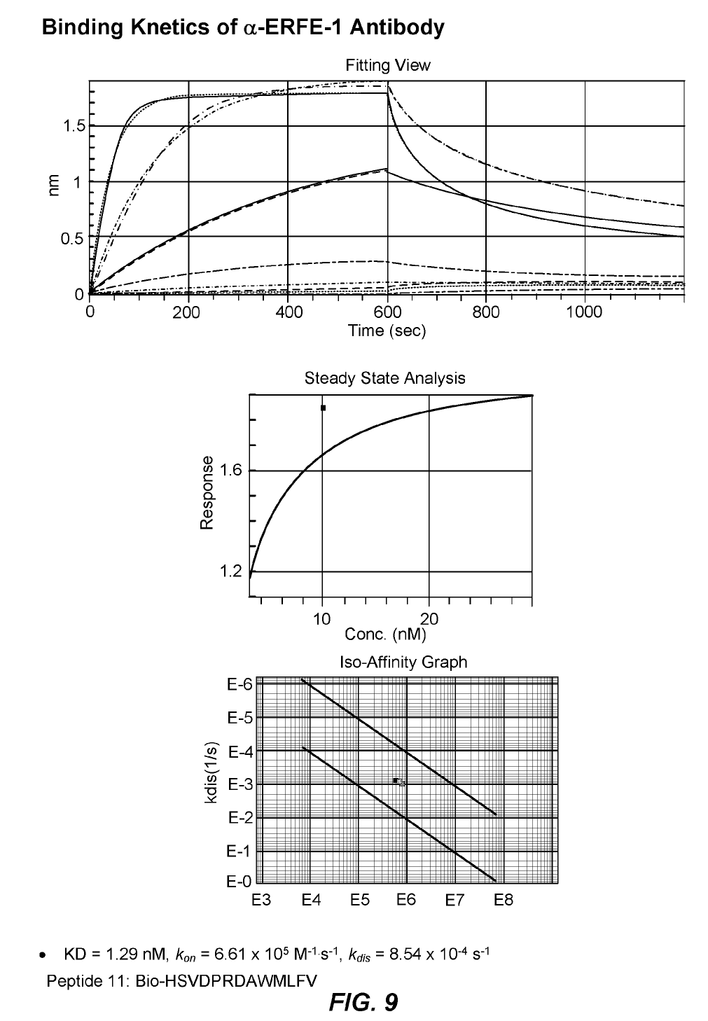

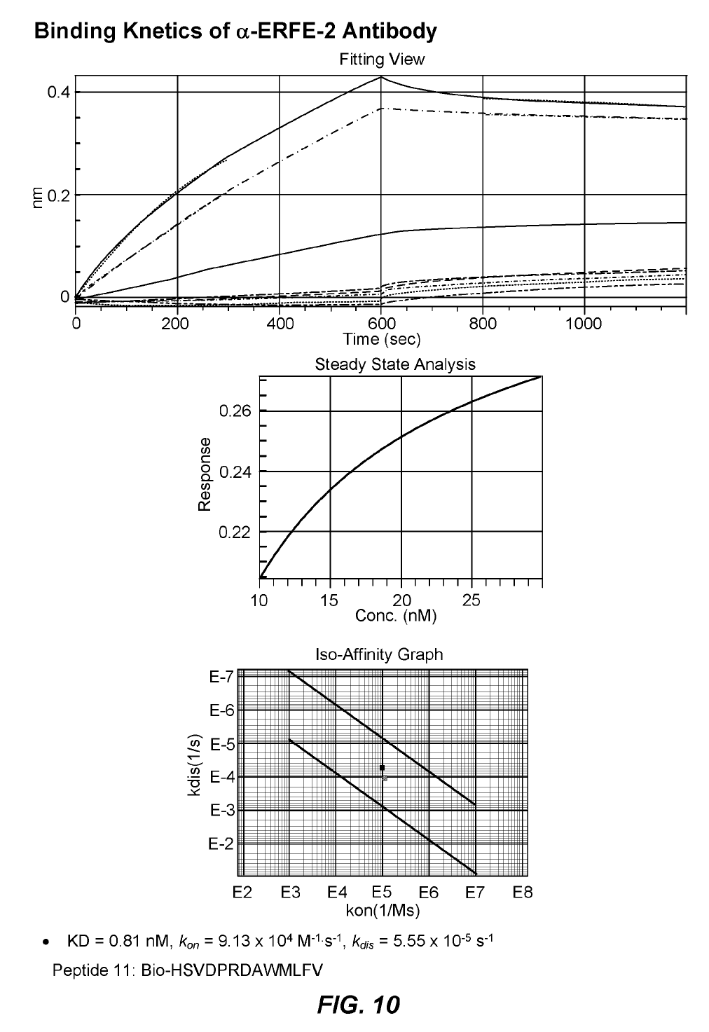

Affinity kinetics was determined on a ForteBio Octet Red96 analyzer. Briefly, a biotinylated 13-mer ERFE peptide captured on streptavidin coated Dip and Read Biosensors for Kinetics (ForteBio) at room temperature in an assay buffer of PBS+0.1% BSA+0.02% Tween-20 pH 7.2. Sensors were washed in assay buffer and then incubated with purified α-ERFE-1 and α-ERFE-2 monoclonal antibodies, respectively, in 3 fold dilution series for 5 minutes in assay buffer to determine association kinetics of the antibody with the peptide. Sensors were then incubated in assay buffer for 10 minutes to determine dissociation kinetics. The resulting kinetics parameters were calculated with ForteBio analysis suite 8.0 using a 1:1 model. Results for these assays are shown in FIG. 9 and FIG. 10.

### Example 5

### ERFE Antibody Generation-Fusion/Screening

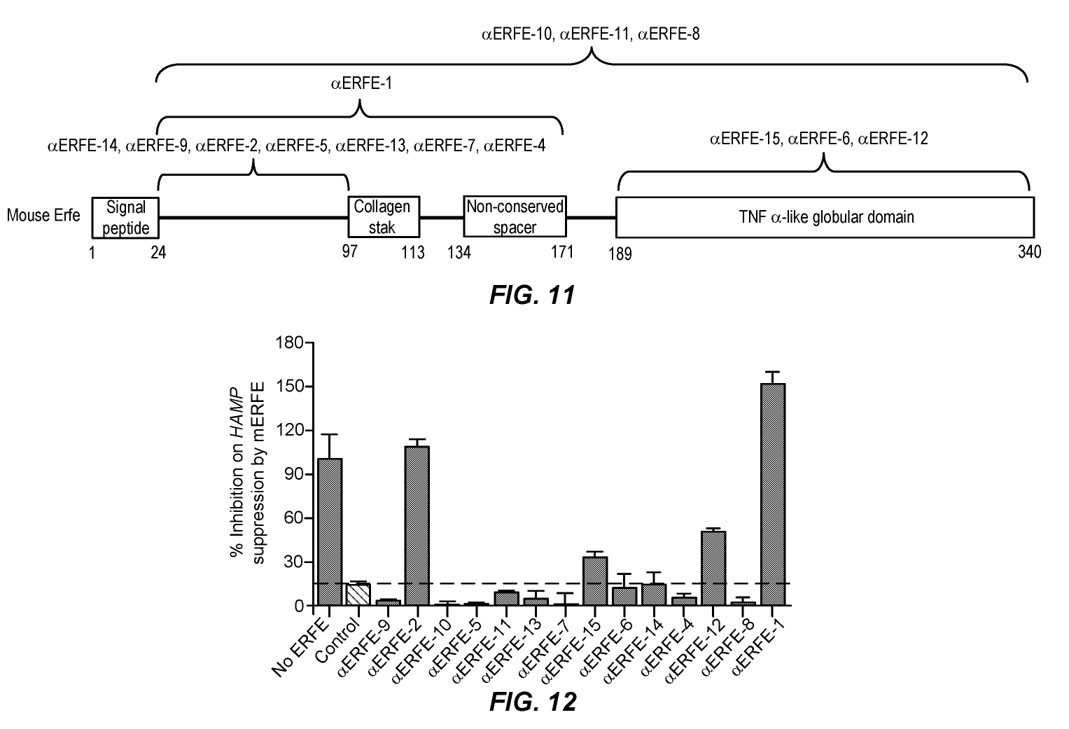

Mice were immunized with Fc-mERFE 24-340 using a modified rapid immunization at multiple sites (RIMMS) protocol, following steps known by those of skill in the art. Following establishment of a high titer response to the immunogen, lymph node B cells were electrofused to mouse myeloma cells. The resulting hybridomas were plated into soft agar, and individual colonies were picked and grown in 96 well plates. Supernatant from approximately 2400 wells was screened for mERFE reactivity. A non-relevant Fc-fusion protein was used to eliminate binders to the human Fc portion of the immunogen. To support screening and characterization of the resulting hybridomas, multiple mouse and human ERFE recombinant proteins containing different putative functional domains of the protein were produced in the HEK293-Freestyle system. Of the 2400 hybridoma clones that were initially screened, 65 were identified that were positive by ELISA for binding to the Fc-mERFE 24-340 (immunogen) and negative for binding to the irrelevant Fc-fusion protein used for counter screening. Upon follow up of these 65 clones, 29 were confirmed to bind to both Fc-mERFE 24-340 (immunogen) and HIS-Flag mERFE 24-340 (confirming specific binding to the mERFE protein) by ELISA at a >10-fold signal over background. Of these 29 clones, 19 were also found to bind to HIS-Flag hERFE by ELISA at a >10-fold signal over background. These 19 mouse/human cross-reactive binders were selected for functional characterization (FIG. 11).

### Example 6

### Functional Antibody Screening

The 19 mouse/human ERFE reactive antibody clones identified in the hybridoma screen were analyzed in the Hep3B hepcidin mRNA (HAMP) cellular assay (FIG. 12). Hybridoma supernatants containing ERFE specific antibodies were tested for their ability to inhibit ERFE-mediated suppression of hepcidin levels by neutralizing the effects of the Fc-mERFE 24-340 protein. To determine neutralization, 500 ng/ml Fc-mERFE 24-340 was pre-incubated with hybridoma supernatant (1:5 dilution) for 30 minutes. The mixture was added to Hep3B cells for 15 hours followed by determination of relative HAMP expression level. Two hybridoma clones were identified that completely blocked the suppressive effect of Fc-mERFE 24-340 on HAMP expression. As this assay was performed with hybridoma supernatants that had not been normalized for antibody concentration, two additional clones that showed low/moderate level of inhibitory activity relative to control hybridoma media were also selected for follow up to account for the potential that their lower activity could be due to lower concentrations of antibody in the samples.

### Example 7

### Identification of Lead Monoclonal Antibodies Clones

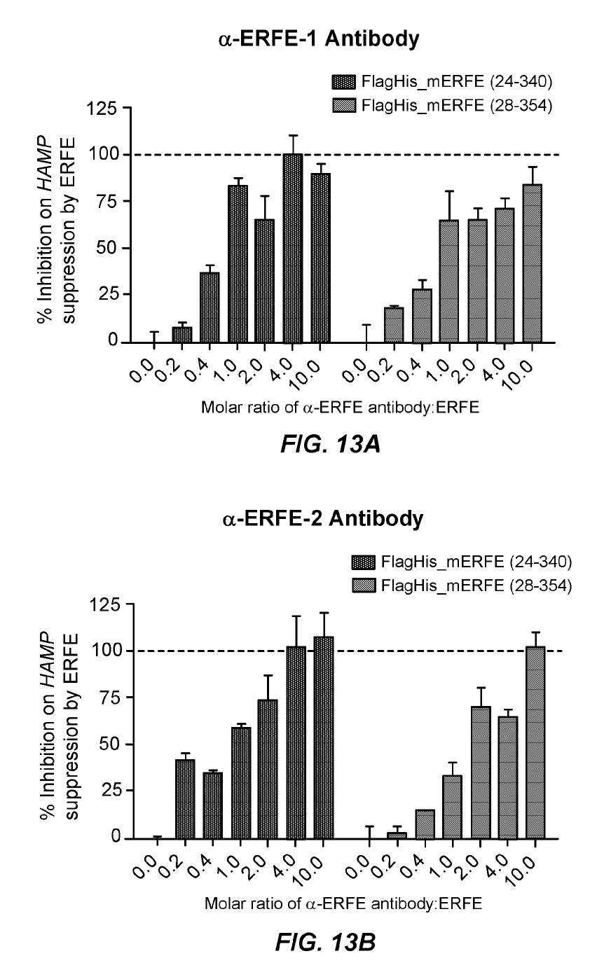

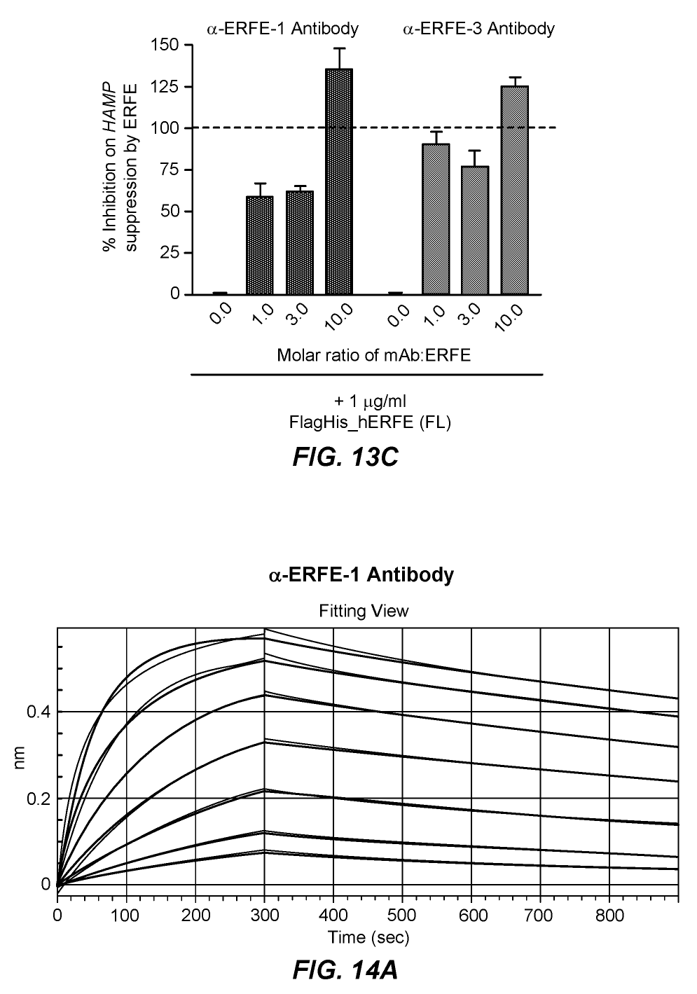

To follow up on the preliminary cellular functional data from the hybridoma supernatants, antibody was purified from the hybridomas, binding confirmed by ELISA, and functional activity determined in the cellular Hep3B hepcidin mRNA (HAMP) suppression assay (FIG. 13A and FIG. 13B). α-ERFE-1, α-ERFE-2, and α-ERFE-3 antibodies were pre-incubated with 500 ng/ml HIS-Flag mERFE 24-340 or HIS-Flag hERFE 28-354, added to Hep3B cells and relative HAMP expression determined after 15-hour incubation. α-ERFE-1, α-ERFE-2, and α-ERFE-3 purified antibody clones demonstrated dose-dependent inhibition of ERFE-mediated HAMP suppression. α-ERFE-1 and α-ERFE-2 antibodies neutralized activity on an approximately equimolar basis against both mouse and human ERFE (albeit to a slightly less potent effect against hERFE 28-354). Similarly, α-ERFE-3 antibody displayed comparable inhibition of ERFE-mediated HAMP suppression relative to α-ERFE-1 antibody, when tested using 1 μg/ml HIS-Flag hERFE 28-354 (FIG. 13C). Two additional purified antibody clones were also tested but these clones demonstrated poor neutralizing activity. HIS-Flag ERFE was tested because it is physiologically relevant.

### Example 8

### Antibody Binding Affinity

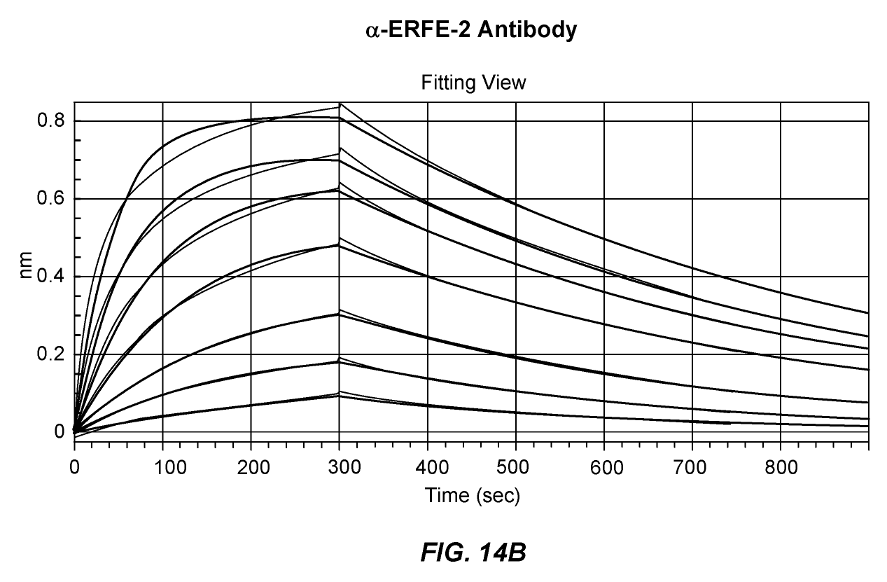

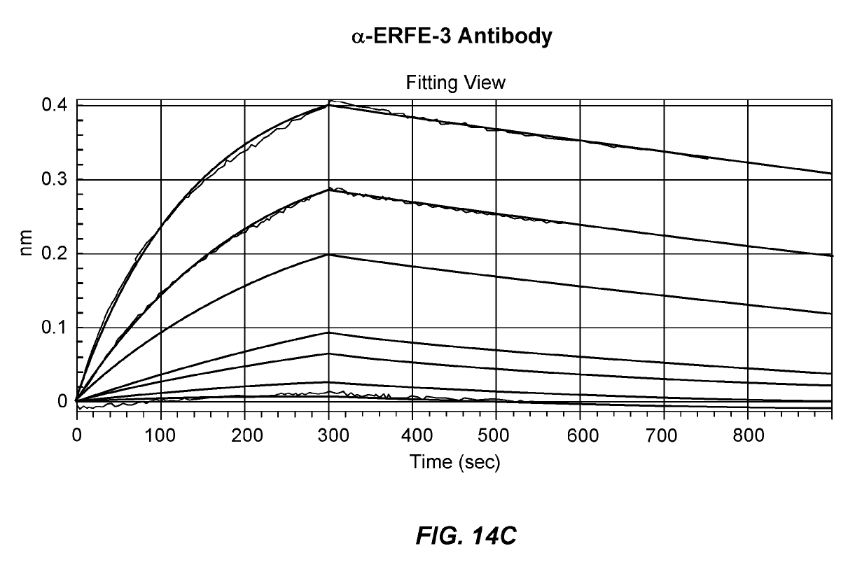

The binding affinity for purified hybridoma antibodies was determined by ForteBio Octet Bio-Layer Interferometry (BLI; FIG. 14A, FIG. 14B, and FIG. 14C) analysis using monovalent human ERFE and summarized in Table 3.

TABLE 3
Binding kinetics and affinity for purified hybridoma antibodies
α-ERFE-1, α-ERFE-2, and α-ERFE-3.

| αERFE Ab | KD (M) | Kon (1/Ms) | Kdis (1/s) | Full X\hat{ }2 | Full R\hat{ }2 |
|---|---|---|---|---|---|
| 1 | 1.84E−09 | 2.81E+05 | 5.12E−04 | 2.34E−02 | 9.96E−01 |
| 2 | 4.01E−09 | 5.01E+05 | 1.98E−03 | 3.14E−02 | 9.97E−01 |
| 3 | 6.67E−09 | 9.23E+04 | 6.21E−04 | 6.64E−03 | 9.98E−01 |

### Example 9

### Cynomolgus Monkey Reactivity

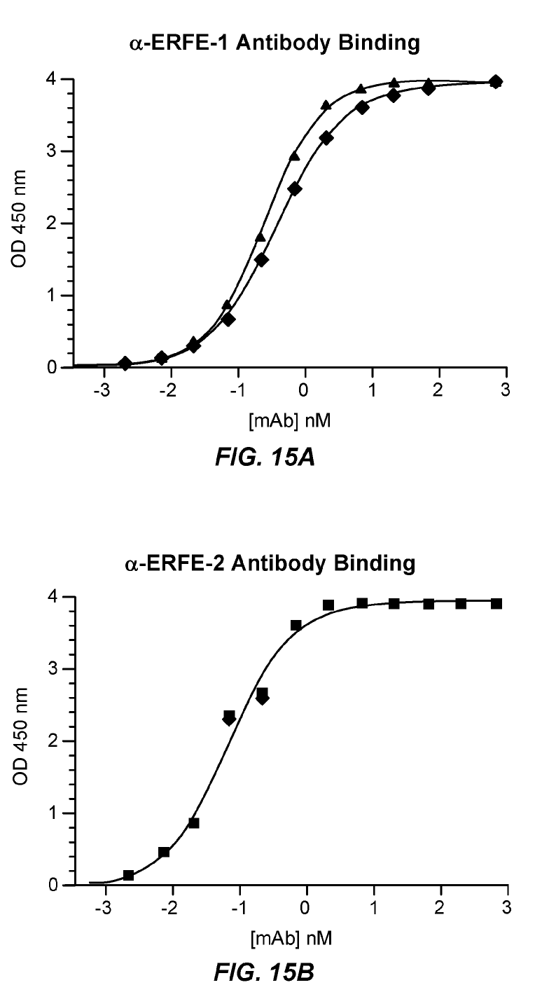

To support preclinical development of an ERFE antibody therapeutic, the homology of cynomolgus (cyno) monkey ERFE and species cross-reactivity of aERFE-1 and aERFE-2 antibodies was examined. On the protein level, ERFE is highly conserved with human and cyno ERFE sharing greater than 92% amino acid identity. There is however a single amino acid difference within the ERFE antibody binding region (cyno ERFE contains a single amino acid change (A to T) in the antibody binding domain (PRDA/TWMLFV (SEQ ID NO: 20)). The gene encoding cyno ERFE was cloned, the resulting protein (HIS-Flag cERFE 28-354) was expressed, purified, and assayed for ERFE antibody binding activity by ELISA (FIG. 15A and FIG. 15B). Both EFRE antibodies were found to have the same apparent affinity to human and cyno ERFE.

### Example 10

### Antibody Specificity

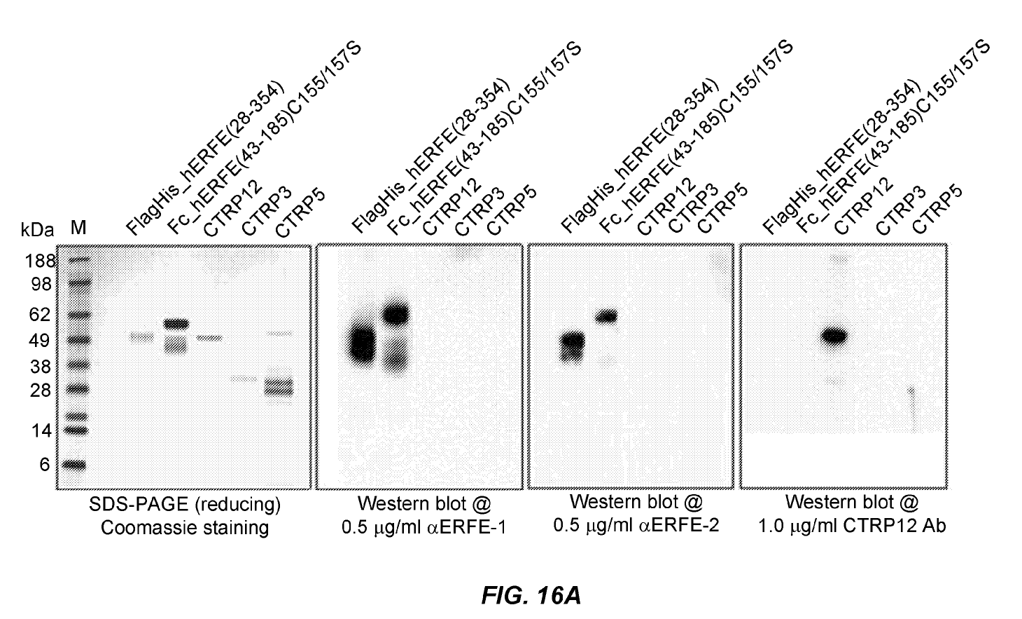

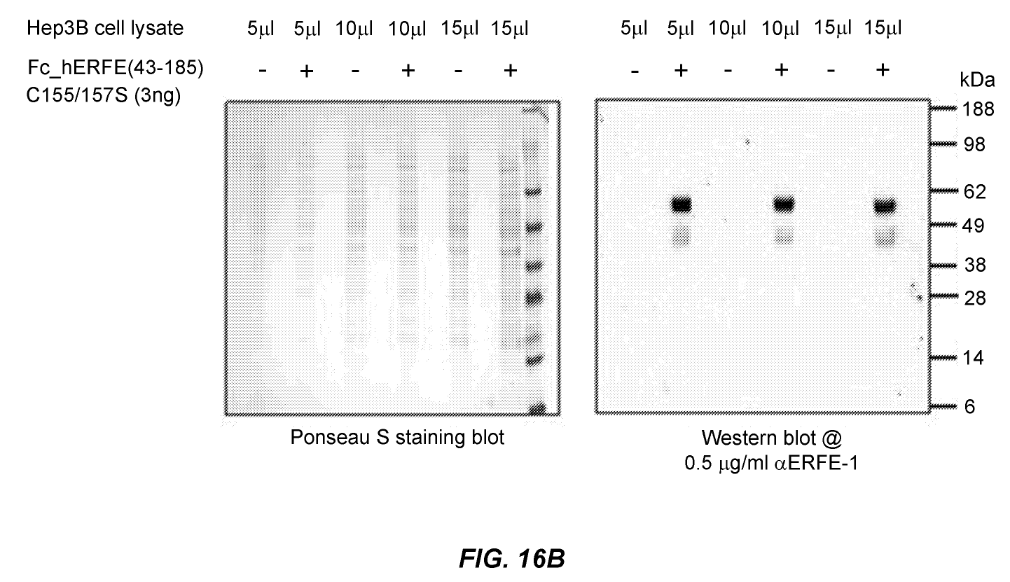

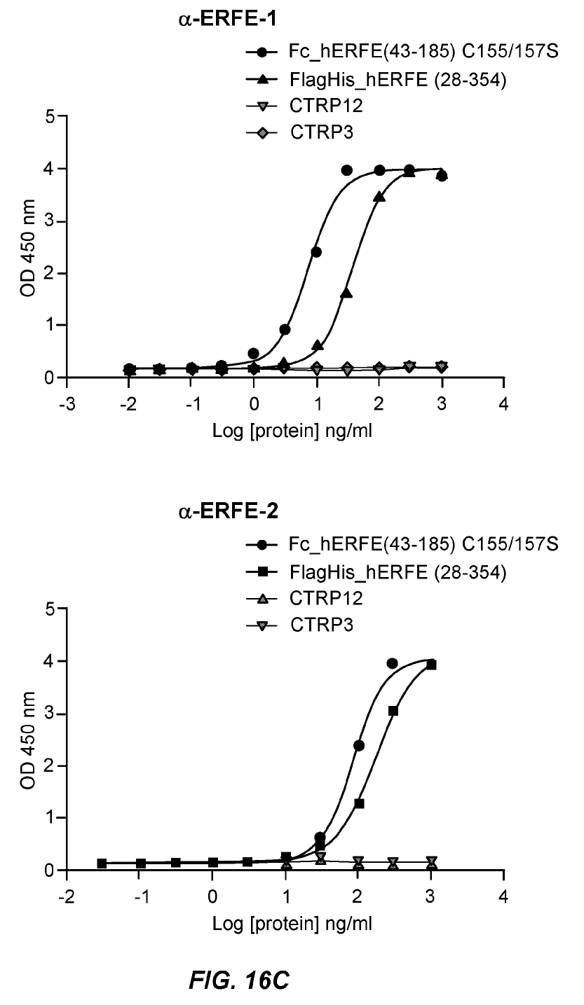

To examine specificity of ERFE hybridoma antibodies, BLAST search was performed to identify proteins with the closest amino acid similarity to ERFE. This included specifically searching the protein sequence database against a peptide sequence contained within the antibody binding epitope. CTRP 12 was identified as the protein with the highest homology to ERFE (CTRP 12 shares 37% overall homology). CTRP 3 and CTRP 5 are from the same protein family and have similar expression patterns to ERFE but share less homology. Antibodies were tested by both western blot and ELISA against a panel of related proteins (CTRP) to determine whether they were specific binders. The antibodies recognized both denatured ERFE protein (western blot) and native ERFE protein (ELISA) but failed to recognize the non-ERFE proteins. In addition, Hep3B lysates were spiked with Fc-hERFE to show that other cellular proteins are not reactive with the antibody (FIG. 16A, FIG. 16B, and FIG. 16C).

### Example 11

### ERFE Antibodies In Vivo Activity

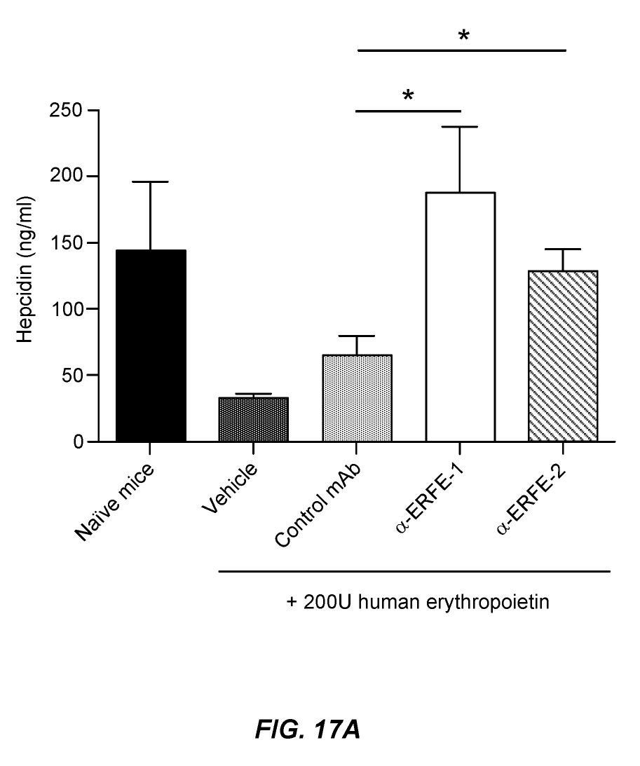

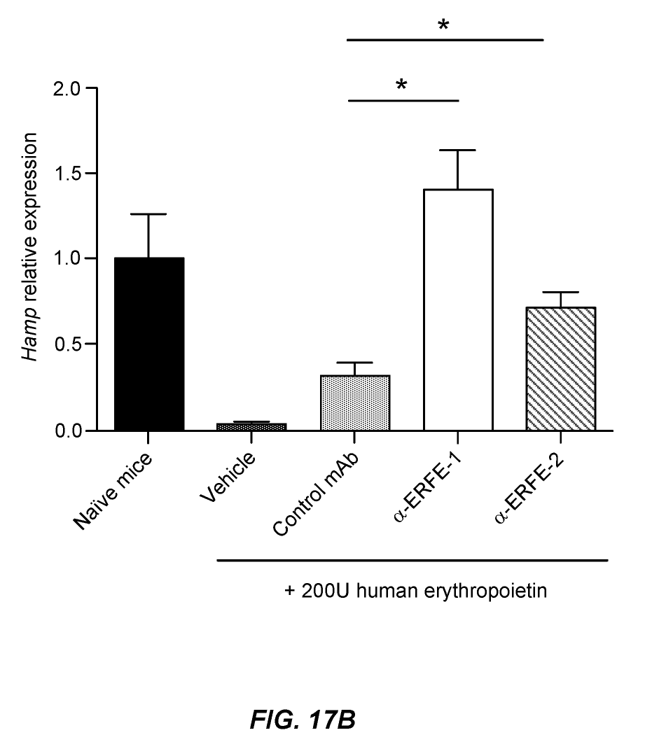

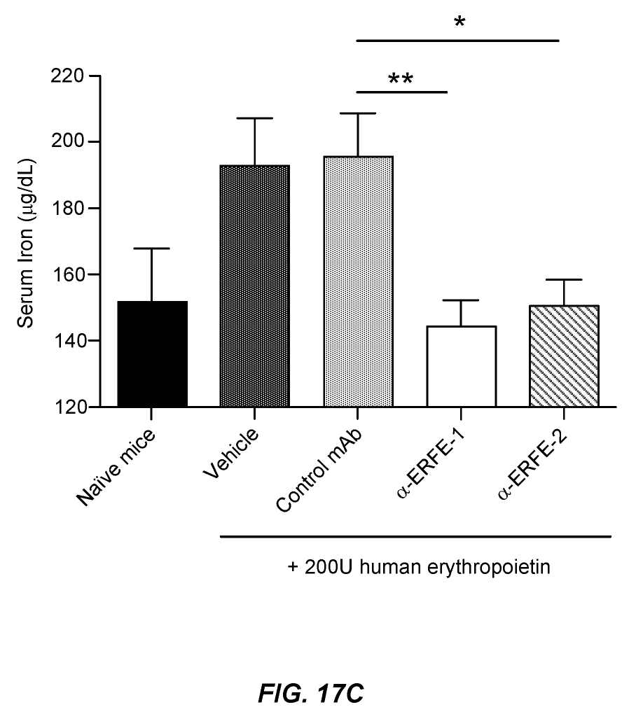

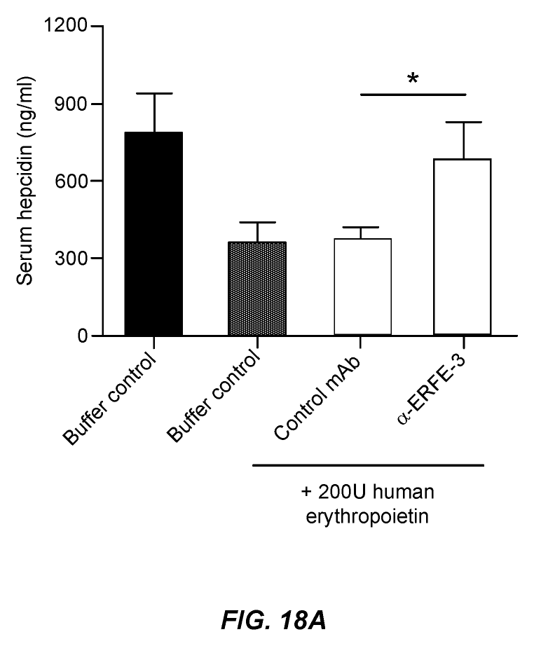

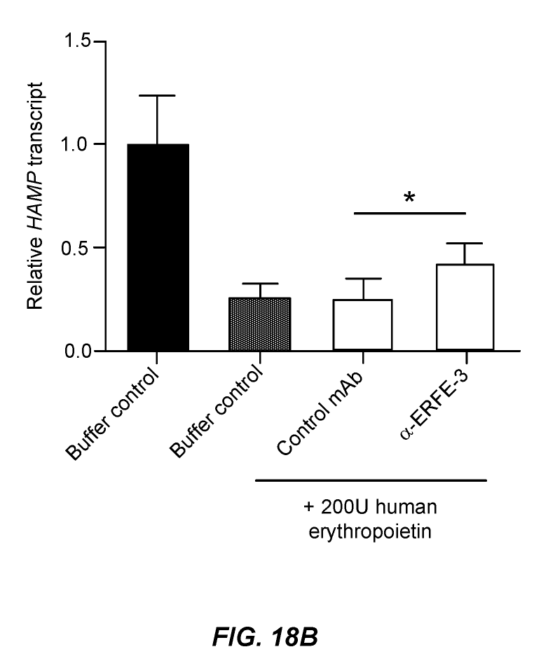

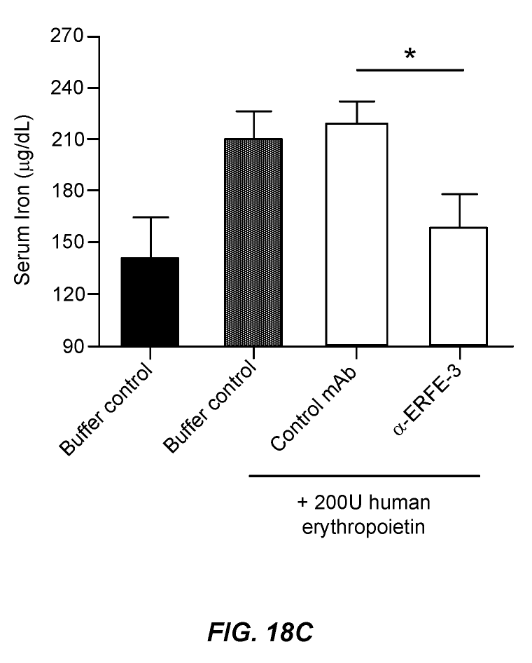

To establish a pharmacokinetics and pharmacodynamics (PK/PD) relationship profile for the lead ERFE antibodies, an in vivo study was conducted. C57BL/6J mice were treated with 10 mg/kg of α-ERFE-1, α-ERFE-2, or α-ERFE-3 by intraperitoneal (IP) injection 24 hours prior to receiving an IP injection of 200 units/mouse of erythropoietin (EPO) used to stimulate erythropoiesis. 16 hours post-EPO injection, liver tissue and serum were harvested for analysis. Mice displayed lowered liver HAMP mRNA expression and serum Hepcidin concentration, along with increased serum Iron levels, in accordance with EPO stimulation of ERFE. Prior treatment with α-ERFE-1 or α-ERFE-2 effectively buffered any change in liver HAMP mRNA expression and serum Hepcidin concentration along with any change in serum iron levels (FIG. 17A, FIG. 17 B, and FIG. 17C). Similarly, prior treatment with α-ERFE-3 also helped to buffer any change in liver HAMP mRNA expression and serum Hepcidin concentration along with any change in serum iron levels (FIG. 18A, FIG. 18 B, and FIG. 18C).

1.-532. (canceled)

533. A method of modulating ERFE polypeptide activity in an individual in need thereof, comprising contacting the ERFE polypeptide with a sufficient amount of an antibody that specifically binds to the ERFE polypeptide or a composition comprising the antibody.

534. The method of claim 533, wherein the antibody that binds to at least one of the following residues of an ERFE polypeptide: D77, P78, R79, D80, A81, W82, M83, L84, F85, or V86 of SEQ ID NO: 2.

535. The method of 533, wherein the antibody binds to an epitope comprising all or part of the sequence DPRDAWMLFV (SEQ ID NO: 1).

536. The method of 533, wherein the antibody comprises an IgG constant domain.

537. The method of 536, wherein the antibody comprises an IgG1, IgG2, IgG3, or IgG4 constant domain, or a variant thereof.

538. The method of 533, wherein the antibody is human.

539. The method of 533, wherein the antibody is humanized.

540. The method of 533, wherein the antibody is chimeric.

541. The method of 533, wherein the antibody partially or completely inhibits erythroferrone activity.

542. The method of 533, wherein the antibody partially or completely inhibits suppression of hepcidin mRNA expression.

543. The method of 533, wherein the antibody has a KD of less than 1.0×10⁻⁸ M.

544. The method of 533, wherein the antibody is a neutralizing antibody.

545. A composition comprising an antibody that specifically binds to an erythroferrone (ERFE) polypeptide and an excipient.

546. The composition of 545, wherein the antibody binds to an epitope comprising all or part of the sequence DPRDAWMLFV (SEQ ID NO: 1).

547. The composition of 545, wherein the antibody partially or completely inhibits erythroferrone activity.

548. The composition of 545, wherein the antibody partially or completely inhibits suppression of hepcidin mRNA expression.

549. The composition of 545, wherein the antibody has a KD of less than 1.0×10⁻⁰⁸ M.

550. The composition of 545, wherein the antibody blocks suppression of hepcidin mRNA expression by an ERFE polypeptide.

551. The composition of 545, wherein the antibody is a neutralizing antibody.

552. The composition of 545, wherein the composition is an isotonic solution.


SEQUENCE LISTING (20 sequences)

SEQ ID NO: 1 - protein, 10 aa, Artificial Sequence
  Description of Artificial Sequence Synthetic peptide
DPRDAWMLFV

SEQ ID NO: 2 - protein, 354 aa, Homo sapiens
MAPARRPAGARLLLVYAGLLAAAAAGLGSPEPGAPSRSRARREPPPGNELPRGPGESRAGPAARPPEPTAERAHSVDPRDAWMLFVRQSDKGVNGKKRSRGKAKKLKFGLPGPPGPPGPQGPPGPIIPPEALLKEFQLLLKGAVRQRERAEPEPCTCGPAGPVAASLAPVSATAGEDDDDVVGDVLALLAAPLAPGPRAPRVEAAFLCRLRRDALVERRALHELGVYYLPDAEGAFRRGPGLNLTSGQYRAPVAGFYALAATLHVALGEPPRRGPPRPRDHLRLLICIQSRCQRNASLEAIMGLESSSELFTISVNGVLYLQMGQWTSVFLDNASGCSLTVRSGSHFSAVLLGV

SEQ ID NO: 3 - protein, 13 aa, Artificial Sequence
  Description of Artificial Sequence Synthetic peptide
GLGVPESAEPVGT

SEQ ID NO: 4 - protein, 13 aa, Artificial Sequence
  Description of Artificial Sequence Synthetic peptide
PESAEPVGTHARP

SEQ ID NO: 5 - protein, 13 aa, Artificial Sequence
  Description of Artificial Sequence Synthetic peptide
EPVGTHARPQPPG

SEQ ID NO: 6 - protein, 13 aa, Artificial Sequence
  Description of Artificial Sequence Synthetic peptide
THARPQPPGAELP

SEQ ID NO: 7 - protein, 13 aa, Artificial Sequence
  Description of Artificial Sequence Synthetic peptide
PQPPGAELPAPPA

SEQ ID NO: 8 - protein, 13 aa, Artificial Sequence
  Description of Artificial Sequence Synthetic peptide
GAELPAPPANSPP

SEQ ID NO: 9 - protein, 13 aa, Artificial Sequence
  Description of Artificial Sequence Synthetic peptide
PAPPANSPPEPTI

SEQ ID NO: 10 - protein, 13 aa, Artificial Sequence
  Description of Artificial Sequence Synthetic peptide
ANSPPEPTIAHAH

SEQ ID NO: 11 - protein, 13 aa, Artificial Sequence
  Description of Artificial Sequence Synthetic peptide
PEPTIAHAHSVDP

SEQ ID NO: 12 - protein, 13 aa, Artificial Sequence
  Description of Artificial Sequence Synthetic peptide
IAHAHSVDPRDAW

SEQ ID NO: 13 - protein, 13 aa, Artificial Sequence
  Description of Artificial Sequence Synthetic peptide
HSVDPRDAWMLFV

SEQ ID NO: 14 - protein, 13 aa, Artificial Sequence
  Description of Artificial Sequence Synthetic peptide
PRDAWMLFVKQSD

SEQ ID NO: 15 - protein, 13 aa, Artificial Sequence
  Description of Artificial Sequence Synthetic peptide
WMLFVKQSDKGIN

SEQ ID NO: 16 - protein, 13 aa, Artificial Sequence
  Description of Artificial Sequence Synthetic peptide
VKQSDKGINSKRR

SEQ ID NO: 17 - protein, 13 aa, Artificial Sequence
  Description of Artificial Sequence Synthetic peptide
DKGINSKRRSKAR

SEQ ID NO: 18 - protein, 13 aa, Artificial Sequence
  Description of Artificial Sequence Synthetic peptide
NSKRRSKARRLKL

SEQ ID NO: 19 - protein, 13 aa, Artificial Sequence
  Description of Artificial Sequence Synthetic peptide
RSKARRLKLGLPG

SEQ ID NO: 20 - protein, 9 aa, Artificial Sequence
  Description of Artificial Sequence Synthetic peptide
  MOD_RES (4)..(4): Ala or Thr
PRDXWMLFV